SEQUENCE LISTING (12 sequences)

SEQ ID NO: 1 - DNA, 18 bp, Artificial Sequence
  primer
acagactgaccgagcgaa

SEQ ID NO: 2 - DNA, 18 bp, Artificial Sequence
  Primer
ctccaggtagactctccg

SEQ ID NO: 3 - DNA, 18 bp, Artificial Sequence
  Primer
gacggggagacacggaaa

SEQ ID NO: 4 - DNA, 18 bp, Artificial Sequence
  Primer
caagagcgcaggtcctct

SEQ ID NO: 5 - DNA, 18 bp, Artificial Sequence
  Primer
cggaatgtgaaggcccag

SEQ ID NO: 6 - DNA, 18 bp, Artificial Sequence
  Primer
cactccacgcacgtgcca

SEQ ID NO: 7 - DNA, 20 bp, Artificial Sequence
  Primer
cactccatgaggtatttctc

SEQ ID NO: 8 - DNA, 18 bp, Artificial Sequence
  Primer
caagagcgcaggtcctct

SEQ ID NO: 9 - DNA, 18 bp, Artificial Sequence
  Primer
cgatattcctcaggtact

SEQ ID NO: 10 - DNA, 18 bp, Artificial Sequence
  Primer
caactttcagcagcttac

SEQ ID NO: 11 - DNA, 20 bp, Artificial Sequence
  Primer
tgctatccctgtacgcctct

SEQ ID NO: 12 - DNA, 20 bp, Artificial Sequence
  Primer
ccatctcttgctcgaagtcc

(19) United States
(12) Patent Application Publication
(10) Pub. No.: US 2021/0153484 A1
(43) Pub. Date: May 27, 2021

(54) Humanized Mouse Model

(71) Applicant: The Wistar lnstitute, Philadelphia, PA (US)

(72) Inventors: Rajasekharan Somasundaram, Philadelphia, PA (US); Kar Muthumani, Philadelphia, PA (US); Meenhard Herlyn, Philadelphia, PA (US)

(21) Appl. No.: 17/045,562

(22) PCT Filed: Apr. 9, 2019
(86) PCT No.: PCT/US19/26552

§ 371 Date: Oct. 6, 2020

Related U.S. Application Data
(60) Provisional application No. 62/655,067, filed on Apr. 9, 2018.

(51) Int. Cl. A01K 67/027; A61K 49/00; C07K 16/30

(52) U.S. Cl. CPC A01K 67/0271; A61K 49/0008; C07K 16/30; A01K 2267/01; A01K 2227/105; A01K 2207/15; A01K 2267/0331; A01K 2207/12

(57) ABSTRACT

The present invention relates to a humanized mouse, methods for generating a humanized mouse, and methods of using the humanized mouse for testing a vaccine, drug or treatment. Also provided are other uses for the humanized mouse.

### CROSS-REFERENCE TO RELATED APPLICATION

This application claims priority to U.S. Provisional Application No. 62/655,067, filed Apr. 9, 2018, which is hereby incorporated by reference in its entirety.

### STATEMENT REGARDING FEDERALLY SPONSORED RESEARCH OR DEVELOPMENT

This invention was made with government support under NIH Grant Nos US4 CA224070 and CA114046-10 awarded by the National Institutes of Health, and under Department of Defense Grant No. PRCRP WX1XWH-16-1-0119 [CA150619]. The government has certain rights in the invention.

### BACKGROUND OF THE INVENTION

Experimental testing of new drugs and of new methods of treatment has traditionally been done on primates such as chimpanzees and on rodents such as rats and mice, due to the technical and ethical constraints placed on conducting clinical trials in human subjects. Several techniques currently exist for the generation of transgenic and other genetically modified mice. Immunological studies are generally conducted on immunodeficient mouse strains, to minimize rejection of transplanted cells or tissues. Efforts have been made to generate humanized mice by transplanting hematopoietic stem cells into immunodeficient mice. However, it has been difficult to control the differentiation of the human stem cells after transplantation into mice.

Immune checkpoint therapy is rapidly emerging as a front-line treatment option for many solid tumors. However, many patients do not respond to anti-PD1 therapy, and some patients show initial responses followed by reemergence of therapy-resistant lesions.

There remains a need for methods and compositions for generating a humanized mouse model that permits tuning of the differentiation and development of human stem cells and thus tuning the immune response in the mouse. There also remains a need for a good pre-clinical model that mimics the human tumor immune environment.

### SUMMARY OF THE INVENTION

Provided is a humanized mouse comprising:

(a) CD34+ cells from human fetal liver and/or human fetal thymus, and

(b) one or more exogenously introduced polynucleotides encoding a cytokine or cytokine receptor.

Also provided is a method of generating a humanized mouse, the method comprising transplanting CD34+ cells from human fetal liver and/or human fetal thymus into an immunodeficient mouse; and delivering one or more polynucleotides encoding a cytokine or cytokine receptor to the mouse, thereby generating the humanized mouse. In some embodiments, the CD34+ cells from human fetal liver and/or human fetal thymus may be transplanted by renal grafting. In further embodiments, the CD34+ cells from human fetal liver and/or human fetal thymus is transplanted under a renal capsule. In some embodiments, when more than one polynucleotides are delivered, the more than one polynucleotides are delivered to the mouse simultaneously or serially. In some embodiments, when the cytokine or cytokine receptor is expressed in the humanized mouse from the one or more polynucleotides, subpopulations of human hematopoietic cells are generated.

Provided is a humanized mouse comprising:

(a) CD34+ cells from human induced pluripotent stem (iPS) cells, and

(b) one or more exogenously introduced polynucleotides encoding a cytokine or cytokine receptor.

In some embodiments, the iPS cells are from fibroblasts or PBMCs that have been reprogrammed. In some embodiments, the fibroblasts or PBMCs have been reprogrammed with OCT4, KLF4, SOX2 or c-Myc.

Also provided is a method of generating a humanized mouse, the method comprising transplanting CD34+ cells from human induced pluripotent stem (iPS) cells into an immunodeficient mouse; and delivering one or more polynucleotides encoding a cytokine or cytokine receptor to the mouse, thereby generating the humanized mouse. In some embodiments, when more than one polynucleotides are delivered, the more than one polynucleotides are delivered to the mouse simultaneously or serially. In some embodiments, when the cytokine or cytokine receptor is expressed in the humanized mouse from the one or more polynucleotides, subpopulations of human hematopoietic cells are generated.

Provided is a method for generating a humanized mouse melanoma model comprising generating a humanized mouse by any one of the methods described above and transplanting HLA-A allele matched melanoma cells into the humanized mouse.

Also provided is a method for measuring an immune response to a melanoma cell comprising administering HLA-A allele matched melanoma cells to the humanized mouse and measuring an immune response to the melanoma cells in the humanized mouse.

Provided is a method for testing a vaccine comprising administering a vaccine to the humanized mouse and measuring an immune response to the vaccine in the humanized mouse.

Provided is a method for testing a drug or a treatment in a humanized mouse comprising administering the drug or treatment to the humanized mouse and measuring an immune response to the drug or treatment in the humanized mouse.

Also provided is a method for generating a polypeptide encoded by an exogenous polynucleotide in a humanized mouse comprising administering exogenous polynucleotide to the humanized mouse. In some embodiments the polypeptide encoded by the exogenous polynucleotide is an antibody or a fragment thereof In further embodiments, the antibody is a monoclonal antibody or fragment thereof. In some embodiments, the antibody or fragment thereof is a Fv, Fab, F(ab)₂, or a single chain antibody (scFv). In further embodiments, the antibody or fragment thereof is a chimeric, human or humanized antibody or fragment thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic representation of the immune cytokines driving development of humanized mice after reconstitution with CD34+ cells from fetal liver of fetal thymus in NSG mice.

FIGS. 2A-2D illustrate in vitro expression of cytokines and cytokine receptors. FIG. 2A is a diagrammatic representation of various immune cytokines constructs cloned into the mammalian expression vector, pMV101. FIG. 2B shows that expression of immune cytokine construct was verified by ELISA of 293T cells transfected with plasmids that express immune cytokines and the levels of cytokines in transfected cells were analyzed by ELISA. FIG. 2C shows Western blot analysis of transfected cells with respective antibody. FIG. 2D shows flow cytometry analysis of cytokine or cytokine-receptor transfected cells. For the flow cytometry analysis, two days post transfection with respective plasmids encoding cytokines, transfected cells were stained with specific IgG (1:100) and then stained with the appropriate secondary conjugated IgGs and subsequently gated for FACS analysis as singlet and live cells. The percent of positive cells is indicated in histograms.

FIG. 3 illustrates the time course of cytokine expression. The concentration of immune cytokines were analyzed at various time periods from hu-Mice mice immunized with the cytokines and cytokine levels were measured by ELISA. Results are the mean±SEMs of 2 to 3 mice per cytokines analyzed in duplicate.

FIGS. 4A-4D illustrate circulating human immune cells in the humanized mouse. FIG. 4A shows that after 8-12 weeks, 20-50% of human CD45+ cells were observed in mouse circulating blood. FIG. 4B shows that physiological levels of T- and B-cells (left panel), and normal human ratio of CD4/CD8 (2.0) were observed (right panel). FIG. 4C shows reconstitution of hu-Mice with human lymphocytes populations after modified novel synthetic plasmid immune cytokines delivery. FIG. 4D shows that a higher human CD45 population was generated.

FIGS. 5A-5I illustrate the generation of Hu-mice. FIG. 5A is a schematic of Hu-mice reconstitution. Six weeks old NSG mice are treated with myelo-depleting drug (busulfan [30 mg/kg]; i.p.) 24 h before they receive purified fetal liver-derived CD34+ cells (1×10⁵; i.v.) and autologous thymus grafts (˜2 mm) under the renal capsule. After grafting, mice receive AAV8 containing hu-cytokine transgenes (2×10⁹; i.v.; 5-6 days after CD34 injection) and 6 days later DNA encoding hu-cytokines (50 μg; i.m.; multiple sites). After day 50, mice are periodically bled (100 μl) and characterized for human immune cells by standard flow cytometry assay using fluorochrome conjugated anti-mouse or anti-human antibodies. FIG. 5B shows the repopulation of human CD45+ cells in circulating blood of reconstituted mice. Mice after 8-12 weeks of human CD34+ cell injection show increased number of human CD45+ cells (p<0.0001) in circulating blood when compared to control non-reconstituted NSG mice. FIG. 5C shows enhanced repopulation of human lymphocytes after AAV8-hu-cytokine transgenes delivery. Significant increase in circulating human CD45+ cells (p<0.002 for days 48 and 72 [closed circles and squares] and p<0.01 for day 112 [closed triangles]) in mice that received AAV8 hu-cytokines (IL3, IL-7 and GM-CSF; right panel) when compared to mice that did not receive hu-cytokines (left panel). FIG. 5D shows myeloid lineage cells after administration of hu-cytokines. CD33+, CD15+, CD11b+ and CD14+ cells are also seen in circulating blood after week 12 of CD34+ cells administration and when mice receive AAV8 hu-cytokines (see above) plus DNA-hu-cytokines (SCF, FLT-3, THPO). FIG. 5E is a graph showing that reconstituted mice show presence of CD56+ NK (innate immune) cells. Mice bled at 10 weeks show increased CD56+ cells that decreases significantly to physiological levels by week 16 (p<0.002). FIGS. 5F-5I show the repopulation of Human T- and B-cells. Generally, by 12-14 weeks physiological levels of human T-and B-cells (FIG. 5F, left panel) and human CD4+ and CD8+ T cells are observed in circulating blood (FIG. 5F, right panel). FIGS. 5G-5I show the repopulation of lymphoid organs with human immune cells. In H&E staining, there is dense repopulation of human lymphocytes in reconstituted mouse thymus (FIG. 5G; right panel) and spleen (FIG. 5H; right panel) when compared to non-reconstituted mouse thymus (FIG. 5G; left panel) and spleen (FIG. 5H; left panel). Dense repopulation of human lymphocytes in mouse kidney (renal capsule) grafted with human thymus FIG. 5I shows all mice were harvested 24 weeks after CD34+ cell injections.

FIGS. 6A-6C illustrate the human immune subpopulation in spleen, thymus, lymph node, small intestines (SI) and lungs of humanized mice. FIG. 6A shows human macrophages in the spleen and SI. Mouse spleen and SI shows the presence of CD68+ monocyte/macrophage lineage cells (left and right panels). FIG. 6B shows human IgA+ and IgE+ cells in the SI and the lungs. Mouse SI and lungs show presence of IgA+ and IgE+ cells (left and right panels) as determined by mouse anti-human IgA or IgG antibodies. Minimal staining of mouse cells was observed when the tissue sections were stained with anti-mouse specific antibodies (See FIG. 23). FIG. 6C shows human CD4+ and CD8+ T-cell subpopulation in lymphoid organs. Mouse spleen (left panels; a), thymus (middle panels) and mesenteric lymph nodes (right panels) show presence of CD4+ (top panels) and CD8+ (bottom panels) T-cells as determined by IHC staining using anti-human CD4 or CD8 antibodies. FIG. 6D shows Human Tγ/δ cells. Reconstituted Hu-mice show presence of T γ/δ cells in the SI, liver, lymph node, skin (data not shown) and spleen (right most panels) of mice that were treated with a bacterial metabolite HMBPP at 50 mg/kg (i.p.). Presence of T γ/δ cells were determined in a IHC staining by using mouse anti-human TcR γ/δ antibody.

FIGS. 7A-7D illustrate cellular immunity in humanized mice. FIG. 7A shows a timeline of DNA immunizations and immune analysis used in the study. NSG-humanized mice were immunized three times, each 2 weeks apart, with 25 μg of pVax1 vector or human telomerase reverse transcriptase (TERT) plasmid and sacrificed 1 week after the 3rd immunization. FIG. 7B is a stacked bar graph. Splenocytes harvested 7 days after the third immunization were incubated with pools of individual human TERT peptides (15-mers overlapping by 11 amino acids). FIG. 7C is a stacked bar graph showing PMA or anti-CD3 stimulation and results Data represent the average numbers of SFUs per million splenocytes from 4 mice/group with values representing the mean responses in each ±SEM. Experiments were performed independently at least two times with similar results. FIG. 7D is a representative ELISpot image from one sample for antigen is shown.

FIGS. 8A-8H illustrate functional characterization of human immune cells in humanized mice. FIGS. 8A-8C show T- and B-cell response to hTERT vaccine. FIG. 8A is a schema for hTERT DNA vaccination. Hu-mice received a total of 3 injections of hTERT vaccine (hTERT DNA [50 μg; i.m] followed by electroporation) every 2 weeks and the mice were sacrificed 1 week after the last injection to determine T- and B-cell responses. FIG. 8B shows anti-TERT T-cell responses. Spleen cells from Hu-mice (n=3) were stimulated overnight (18 h) with pools of overlapping hTERT peptides (15 mer; 2 μg/ml/peptide) spanning the entire hTERT protein. Human IFNγ was detected in ELISPOT assay using a kit. Data are represented as SFU (spot forming units; mean±SE) per 10⁶ splenocytes. hTERT-specific T-cell (IFNγ) response from vaccinated mice was compared to Hu-mice that received pVax1 as control or untreated NSG mice controls. FIG. 8C shows anti-TERT antibody (IgG) responses. Endpoint binding titer was determined in sera of hTERT vaccinated mice after 3 immunizations and compared to sera from NSG mice as controls. FIGS. 8D-8H illustrate the functional ability of immune T-cells to restrict tumor growth. FIG. 8D is a schematic for Hu-mice tumor challenge experiment. FIG. 8E illustrates that Hu-mice with T-cell reconstitution can restrict tumor growth of HLA-A2 matched A375 melanoma cells. Hu-mice that have ˜15% circulating CD8+ cells (closed circle) when challenged with melanoma cells (10⁵; s.c.) can restrict tumor growth significantly (p=0.0281) when compared to non-reconstituted NSG mice (closed circle, top line) and Hu-mice with high circulating B-cells (>65% CD20+; open circles, middle line) have unrestricted tumor growth. Tumor growth measurements are recorded using digital caliper by an independent researcher. FIGS. 8F-8H illustrate that treatment with anti-PD1 antibody can restrict tumor growth of melanoma cells. f. Schema for anti-PD1 therapy. Hu-mice with ˜25% CD45+ cells were randomized, and they receive melanoma cells (10⁵; s.c.). When tumors are palpable, mice receive anti-PD1 (10 mg/kg; i.p. injections) every week for 4 injections and tumor growth measurements are recorded. FIGS. 8G and 8H illustrate that anti-PD therapy restricts melanoma growth. Anti-PD1 antibody can restrict tumor growth of 2 different melanoma cells (WM3629 [HLA-A3]; FIG. 8G and A375 [HLA-A2]; FIG. 8H) significantly (bottom line, closed circles; p<0.05) when compared to Hu-mice treated with control IgG (middle line; open circles) or non-reconstituted NSG mice (top line; closed circles) treated with ant-PD1 antibody. Unrestricted tumor growth in presence of anti-PD1 antibody was observed when Hu-mice were challenged with an aggressive phenotype of tumor (See FIG. 25).

FIGS. 9A-9D illustrate the humoral response against immune antigen. (FIGS. 9A and 9C) Groups of hu-Mice mice were injected with vaccine targets, and serum was collected at one week after second immunization. Individual sera were assessed for antigen-specific IgG content by ELISA analyses. Each bar represents the serum value for an individual animal. (FIGS. 9B and 9D) Target vaccine was transfected in 293T cells and were lysed 48 hours post transfection and subjected to Western blot using immune sera that were raised in mice. Blocking overnight at 4° C. followed by 2 hours at room temperature (1:100 dilution) with primary antibody incubation. Both membranes were finally incubated in 1:5000 secondary antibody (Goat anti-Human IgG) for 1 hour. The blots were then washed and the membranes were imaged on the Odyssey infrared imager (LI-COR). Lane 1 contains the protein molecular weight markers (kDa).

FIG. 10 illustrates induction of human IgA+in Hu-NSG mice+cytokine delivery. The specific serum IgA anti-vaccine antibodies obtained the different routes as indicated in mice that received the targeted immunization and were assessed by ELISA. The standard errors are as shown. Specific human IgA binding Ab responses are shown after two immunizations in Hu-Mice.

FIG. 11 illustrates induction of human antibodies in Hu-NSG mice against a human tumor antigen. ELISA plates were coated with hTERT transfected 293T cell lysates and primary antibody was used from immune sera from hTERT vaccinated (1:50) mice. The second antibody:either anti human IgG-HRP (left) (1:10000) or anti mouse IgG-HRP (right) (1:6000) was added and measured by ELISA analysis. OD, optical density. Seroconversion and specificity of human responses in Hu-Mice.

FIG. 12 illustrates the human mouse melanoma model. Cell lines or PDX are typed for HLA-A1, -A2 or -A3 alleles. The tumor HLA A allele is matched with donor CD34+ cells and the tumor cells are injected into a humanized mouse.

FIG. 13 illustrates an autologous humanized mouse melanoma model (iPS).

FIG. 14 illustrates restricted tumor growth in the presence of CD8+ T-Cells (Hu-Mice)

FIG. 15 illustrates human CD4+ and CD8+ cells in tumors, as well as human CD33+ and CD15⁺ cells in tumors.

FIG. 16 illustrates the restriction of tumor growth after anti-PD1 treatment in Hu-mice.

FIG. 17 illustrates the restriction of tumor growth after anti-PD1 treatment in Hu-mice.

FIG. 18 illustrates no restriction of tumor growth after anti-PD1 treatment in some Hu-mice.

FIGS. 19A-19L illustrate immune and tumor heterogeneity as possible cause of therapy resistance to anti-PD1. FIGS. 19A-19D illustrate the heterogeneous distribution of leukocytes and immune cells in tumors after PD1 treatment. FIG. 19A shows tumor bearing Hu-mice that received anti-PD1 as in FIG. 8F, showed dense leukocyte infiltration of leukocytes (left panel) when compared to mice that received control mouse IgG (right panel) as determined by H&E staining. FIG. 19B shows tumor bearing Hu-mice that received anti-PD1 showed either low to moderate (left panel) or robust (right panel) tumor-infiltration of CD4+ (brown) and CD8+ (blue) T cells within the same tumor. FIG. 19C shows MassCyTOF staining shows heterogeneous and higher distribution of CD8+ T cells (magenta) within the nestin+tumor cells (dark blue) in anti-PD1 treated tumor-bearing mice (lower panels) as compared to low infiltration of CD8+ T-cells (upper panels) in untreated Hu-mice. Distribution of GrB+ T-cells (arrows; 2^{nd} to right bottom panel) was heterogeneous as they were higher on the bottom half of the tumor section when compared to remainder of other nestin+tumor cell areas. FIGS. 19D-19E show that CD8+ T-cells are of memory phenotype as they stain for CD45RO (light blue; left most panel) and areas not infiltrated by CD8+ T-cells reveals the presence of CD4+/FOXP3+ cells (arrows; 2^{nd} panel from left). Down modulation of HLA class I (white arrows) was observed in tumor areas that has higher FOXP3+ cells (middle panel; FIG. 19E). FIGS. 19F-19I show the increase in mast cells after anti-PD1 therapy. CIBERSORT analysis of the RNA-seq data set showed higher expression of mast related genes in tumors obtained from Hu-mice after anti-PD1 treatment (FIG. 19F) and the presence of mast cells was further confirmed by mast cell tryptase IHC staining (FIG. 19G, right panel). Untreated tumors had negligible staining for mast cells (FIG. 19F, left panel). Representative sample of melanoma patient's tumor section also shows the presence of mast cells (FIG. 19H; FIG. 26). CIBERSORT analysis of two independent data sets obtained in melanoma patients showed higher expression of mast cells related genes when compared to pre-therapy tumors (FIG. 19I). FIG. 19J. Co-localization of FOXP3+ T-reg and mast cells after anti-PD1 therapy. Co-localization of these cells as determined by IHC staining suggesting cross-talk. FIG. 19K shows complete regression of tumors after combination of Sunitinib and anti-PD therapy. Established tumors in Hu-mice (as in FIGS. 8G and 8H) were treated with Sunitinib (20 mg/kg) daily by oral gavage and after 72 h, anti-PD1 therapy (10 mg/kg) was given weekly for a total of 6 injections. Complete tumor regression was observed in presence of combination therapy (black inverted triangle; p<0.0001), while Sunitinib alone (grey circles, second line from the bottom), anti-PD1 alone (closed circles, third line from the bottom) or control IgG (open circles, fourth line from the bottom) did not have any effect of tumor growth. FIG. 19L is a graph showing percent of survival of Hu-mice treated with control IgG indicating that treatment with Sunitinib and anti-PD1 increased survival. FIG. 19M is a schematic showing mast cell induced resistance mechanism to anti-PD1.

FIGS. 20A-20B illustrate vector maps. FIG. 20A is a schematic of AAV8 DNA encoding hu-cytokine transgenes. FIG. 20B is a schematic of pMV101 DNA encoding hu-cytokine transgenes.

FIG. 21 illustrates the stability of Hu-mice. FIG. 21 is a graph showing representative examples of Hu-mice batches with longevity of 30 weeks or more after human CD34+ cell injections.

FIGS. 22A-22C illustrate the higher repopulation of human B-cells as compared to T-cells. FIG. 22A shows that reconstituted humanized mice showed increased levels of B-cells than T-cells (p<0.0001) during early phase (8-10 weeks) of human lymphocyte reconstitution. FIGS. 22B and 22C show human CD45+ cells in reconstituted mouse thymus and spleen. Human CD45+ cells (brown staining) are seen in lymphoid organs of mouse thymus (FIG. 22B) and spleen (FIG. 22C) as determined by IHC staining using anti-human CD45 antibody. All mice were harvested 24 weeks after CD34+ cell injections.

FIG. 23 shows control antibody staining for anti-human IgA and IgE. Hu-mice SI and lungs showed minimal staining with anti-mouse specific antibodies.

FIG. 24 human Tα/β expression. TcR sequence analysis of spleen and tumor cells obtained from Hu-mice melanoma model showed diverse expression of T α/β chains in the spleen when compared to more restricted usage in tumors (top panel). TcR α/β chain expression showed high prevalence of several unique VJ clonotypes in tumors (bottom panel).

FIG. 25 shows that treatment with anti-PD1 has no effect on aggressively growing melanoma tumor. In an established Hu-mice melanoma model (see FIG. 8F) anti-PD1 treatment was unable to restrict tumor growth of 451LU.

FIG. 26 illustrates an increase in mast cells in melanoma patients' tumor after anti-PD1 therapy. Representative immunostaining of tumor from human melanoma patients (see FIG. 19G) showed increased presence of mast cells after anti-PD1 therapy (right panel) when compared to untreated individuals (left panel).

FIGS. 27A-27E illustrate changes in the level of chemokines, chemokine receptors and HLA class I after anti-PD1 therapy. FIG. 27A shows RNA seq-analysis of tumors from Hu-mice treated with anti-PD1, which showed high expression of chemokines that are known to bind to CXCR2 and CXCR3 and that are expressed by mast cells. FIG. 27B shows melanoma cells co-express CXCL10. Tumor cells were co-stained with anti-melanoma (HMB45 [dark grey] and anti-human CXCL10 (light grey; white arrows) antibodies. FIGS. 27C-27D show that mast cells co-express CXCR2 and CXCR3. Mast cells were co-stained with anti-MCT (dark grey) and anti-human CXCL10 (light grey; white arrows) antibodies. FIG. 27E shows that downmodulation of HLA class I. HLA class I molecules as determined by staining with anti-HLA class I antibody (light grey) were downmodulated in tumor areas (black arrows) that were infiltrated by mast cells (dark grey).

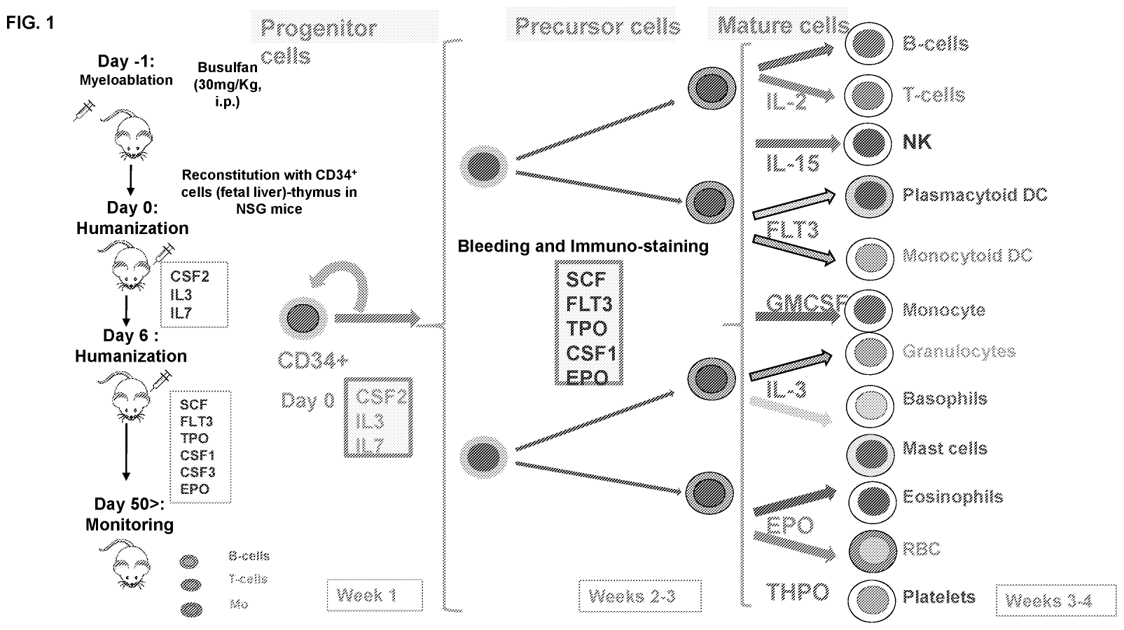

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting. The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g.,sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

The term “immunodeficient” as used herein means lacking the ability to mount an effective immune response to an agent, for example but not limited to, being susceptible to infection. Mice with severe combined immunodeficiency (SCIDs) are often used in research.

The term “NSG™ mouse” as used herein, is a type of immunodeficient mouse that is used in biomedical research (Pearson T, et al. 2008. Creation of “humanized” mice to study human immunity. Curr Protoc Immunol. May; Chapter 15: Unit 15.21; Shultz L D et al. 2005. J. Immunol. 174(10):6477-89). NSG™ mice are commercially available from the Jackson Laboratory or they may be prepared by known methods (Shultz L D et al. 2005. J. Immunol. 174(10):6477-89). For example, NSG™ mice can be generated by backcross matings of C57BL/6J-gnull mice with NOD/SCID mice nine times. NSG™ mice lack functional T and B cells and have reduced macrophage function. NSG mice lack NK cell or NK activity, and have reduced dendritic function. NSGTM mice have a higher level of xenograph engraftment than NOD/SCID mice or beta2-microglobulin deficient NOD/LtSc-SCID (NOD/SCID/beta2m null) mice.

As used herein, “induced pluripotent stem cell” or “iPS cell” refers to a pluripotent stem cell that is generated from adult cells, such as T cells. The expression of reprogramming factors, such as Klf4, Oct3/4 and Sox2, in adult cells convert the cells into pluripotent cells capable of propagation and differentiation into multiple cell types.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “humanized mouse” refers to an immunocompromised mouse engrafted with human haematopoietic stem cells or tissues, or a mouse that transgenically expresses human genes.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Provided is a humanized mouse and methods of generating a humanized mouse. Also provided are methods for generating a humanized mouse melanoma model, as well as methods of testing a vaccine, a drug or a treatment in the humanized mouse.

### Humanized Mouse

Provided is a humanized mouse comprising:

(a) CD34+ cells from human fetal liver and/or human fetal thymus, and

(b) one or more exogenously introduced polynucleotides encoding a cytokine or cytokine receptor.

Also provided is a method of generating a humanized mouse, the method comprising transplanting CD34+ cells from human fetal liver and/or human fetal thymus into an immunodeficient mouse; and delivering one or more polynucleotides encoding a cytokine or cytokine receptor to the mouse, thereby generating the humanized mouse. In some embodiments, the CD34+ cells from human fetal liver and/or human fetal thymus may be transplanted by renal grafting. In further embodiments, the CD34+ cells from human fetal liver and/or human fetal thymus is transplanted under a renal capsule. In some embodiments, when more than one polynucleotides are delivered, the more than one polynucleotides are delivered to the mouse simultaneously or serially. In some embodiments, when the cytokine or cytokine receptor is expressed in the humanized mouse from the one or more polynucleotides, subpopulations of human hematopoietic cells are generated.

Provided is a humanized mouse comprising:

(a) CD34+ cells from human induced pluripotent stem (iPS) cells, and

(b) one or more exogenously introduced polynucleotides encoding a cytokine or cytokine receptor.

In some embodiments, the iPS cells are from fibroblasts or PBMCs that have been reprogrammed. In some embodiments, the fibroblasts or PBMCs have been reprogrammed with OCT4, KLF4, SOX2 or c-Myc.

Also provided is a method of generating a humanized mouse, the method comprising transplanting CD34+ cells from human induced pluripotent stem (iPS) cells into an immunodeficient mouse; and delivering one or more polynucleotides encoding a cytokine or cytokine receptor to the mouse, thereby generating the humanized mouse. In some embodiments, when more than one polynucleotides are delivered, the more than one polynucleotides are delivered to the mouse simultaneously or serially. In some embodiments, when the cytokine or cytokine receptor is expressed in the humanized mouse from the one or more polynucleotides, subpopulations of human hematopoietic cells are generated.

The following description of embodiments applies to both the humanized mice and to the methods of generating a humanized mouse.

In some embodiments, the humanized mouse is an NSG™ mouse. The NSG mouse is a non-obese diabetic (NOD) mouse which is double homozygous for the severe combined immune-deficient (SCID) mutation. A SCID mutation is one that results in deficiencies of T and B cells, resulting in an immunodeficient mouse. SCID mice have defects in the rearrangement of the B cell receptor (BCR) and of the T cell receptor (TCR). Thus, SCID mice are deficient in functional T and B cells. The NSG™ mouse also has the interleukin 2Rgamma allelic mutation (gamma null, or γnull). NSG mice are also known as NOD/SCIDγnull mice or NOG/SCID IL-2RγKO mice.

Following expression of a cytokine from the one or more exogenously introduced polynucleotides, subpopulations of human hematopoietic cells are induced in the humanized mouse. The subpopulations of human hematopoietic cells may comprise T cells, B cells, NK cells, monocytes, dendritic cells, or combinations thereof. The cytokine or cytokine receptor may be human, mouse, recombinant or combinations thereof. In some embodiments, the cytokine or cytokine receptor is recombinant. the cytokine or cytokine receptor is at least one of: Colony stimulating factor 2 (CSF2), Interleukin-3 (IL3), Interleukin-7 (IL7), Stem cell factor (SCF), Fms Related Tyrosine Kinase 3 (FLT3), Thrombopoietin (TPO), Colony stimulating factor 1 (CSF1), Colony stimulating factor 3 (CSF3), Erythropoietin (EPO), Interleukin-15 (IL15), c-kit, or combinations thereof.

### Melanoma Model

Provided is a method for generating a humanized mouse melanoma model comprising generating a humanized mouse by any one of the methods described above and transplanting HLA-A allele matched melanoma cells into the humanized mouse.

Also provided is a method for measuring an immune response to a melanoma cell comprising administering HLA-A allele matched melanoma cells to the humanized mouse and measuring an immune response to the melanoma cells in the humanized mouse.

In the humanized mouse melanoma model, in some embodiments, the melanoma cells are from a cell line. In some embodiments, the melanoma cells are from a patient derived xenograft. In some embodiments, the melanoma cells are from a live tumor bank. In further embodiments, the melanoma cells are typed and matched for HLA-A1, HLA-A2 or HLA-A3 alleles.

In some embodiments, melanocytes are generated from an adult melanoma patient' sfibroblasts using iPS technology. iPS generated melanocytes derived from multiple donors may show different susceptibility to UV irradiation in 3D skin reconstructs. Skin reconstructs comprising melanocytes or early melanoma lesions grafted into humanized mice offer a powerful tool to understand the process of malignant transformation and early melanoma progression.

### Vaccines

Provided is a method for testing a vaccine comprising administering a vaccine to the humanized mouse and measuring an immune response to the vaccine in the humanized mouse.

In some embodiments, the vaccine is specific for a disease that may include, but is not limited to:

(a) a viral disease, such as genital warts, common warts, plantar warts, hepatitis B, hepatitis C, herpes simplex virus type I and type II, molluscum contagiosum, variola, HIV, CMV, VZV, Zika virus, rhinovirus, adenovirus, coronavirus, influenza, para-influenza;
(b) a bacterial disease, such as tuberculosis, and mycobacterium avium, leprosy;
(c) other infectious disease, such as a fungal disease, e.g., candida, aspergillus, or a disease caused by chlamydia, or cryptococcal meningitis, pneumocystis carnii, cryptosporidiosis, histoplasmosis, toxoplasmosis, trypanosome infection, leishmaniasis;
(d) a neoplastic disease, such as intraepithelial neoplasias, cervical dysplasia, actinic keratosis, basal cell carcinoma, squamous cell carcinoma, hairy cell leukemia, Karposi's sarcoma, melanoma, renal cell carcinoma, myelogeous leukemia, multiple myeloma, non-Hodgkin's lymphoma, cutaneous T-cell lymphoma, and other cancers;
(e) a TH-2 mediated, atopic, and autoimmune disease, such as atopic dermatitis or eczema, eosinophilia, asthma, allergy, allergic rhinitis, systemic lupus erythematosis, essential thrombocythaemia, multiple sclerosis, Ommen's syndrome, discoid lupus, alopecia areata, inhibition of keloid formation and other types of scarring, and enhancing would healing, including chronic wounds.

In some embodiments, the vaccine is human TERT.

The vaccine may be administered to the humanized mouse subcutaneously, intraperitoneally or nasally, or by any acceptable route of administration suitable for the disease being targeted.

The vaccine comprises an antigen and optionally, an adjuvant.

In some embodiments, the vaccine is a DNA vaccine. In further embodiments, the DNA vaccine comprises a polynucleotide that encodes an antigen, a polypeptide or a fragment thereof.

### Testing Drug or Treatment

Provided is a method for testing a drug or a treatment in a humanized mouse comprising administering the drug or treatment to the humanized mouse and measuring an immune response to the drug or treatment in the humanized mouse.

In some embodiments, the drug or treatment is for treating a cancer, a viral disease, a bacterial disease, a fungal disease or a parasitic disease. In some embodiments, the drug or treatment comprises an immune checkpoint inhibitor. In further embodiments, the immune checkpoint inhibitor is anti-PD1 or anti-PDL1. In some embodiments, the immune checkpoint inhibitory therapy comprises administration of anti-PD1 antibody or anti-PDL1 antibody to the humanized mouse

In some embodiments, tumor-bearing humanized mice treated with anti-human PD-1 show robust infiltration of T-cells and enhanced restriction of tumor growth.

### Measuring Immune Response

An immune response to any of the methods or treatments described herein may be measured by any of the means that are known to a person of skill in the art. In some embodiments, an immune response is measured by detecting cytokine production. In some embodiments, cytokine production is measured in spleen cells. In further embodiments, the spleen cells are harvested.

In some embodiments, cytokine production is detected by RNA extraction followed by reverse transcription and quantitative PCR. In further embodiments, the PCR is real-time PCR.

In some embodiments, cytokine production is detected by immunoassay. In further embodiments, cytokine production is detected by ELISA.

In some embodiments, cytokine production is compared to a control. In some embodiments, the control is from a humanized mouse that has not received the drug or treatment.

In some embodiments, an immune response is measured by detecting tumor leukocyte infiltration. In some embodiments, tumor infiltrating leukocytes are detected and compared to a control.

### DNA Encoded Protein Synthesis

Provided is a method for generating a polypeptide encoded by an exogenous polynucleotide in a humanized mouse comprising administering exogenous polynucleotide to the humanized mouse. In some embodiments the polypeptide encoded by the exogenous polynucleotide is an antibody or a fragment thereof In further embodiments, the antibody is a monoclonal antibody or fragment thereof. In some embodiments, the antibody or fragment thereof is a Fv, Fab, F(ab)₂, or a single chain antibody (scFv). In further embodiments, the antibody or fragment thereof is a chimeric, human or humanized antibody or fragment thereof.

In some embodiments, the polynucleotide is a plasmid or a vector. In some embodiments, the polynucleotide is DNA. In some embodiments, the polynucleotide is RNA that can be reverse transcribed into DNA. In some embodiments, the vector is a viral vector. Examples of viral vectors include, but are not limited to, Sendai viral vectors, adenoviral vectors, adeno-associated virus vectors, retroviral vectors, lentiviral vectors, and the like.

### Antibodies

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Alternatively, in some embodiments, a non-human antibody is humanized, where specific sequences or regions of the antibody are modified to increase similarity to an antibody naturally produced in a human. In one embodiment, the antigen binding domain portion is humanized.

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A “humanized” antibody retains a similar antigenic specificity as the original antibody. However, using certain methods of humanization, the affinity and/or specificity of binding of the antibody for human CD3 antigen may be increased using methods of “directed evolution,” as described by Wu et al., J. Mol. Biol., 294:151 (1999), the contents of which are incorporated herein by reference herein in their entirety.

In one embodiment, the antibody is a synthetic antibody, human antibody, a humanized antibody, single chain variable fragment, single domain antibody, an antigen binding fragment thereof, and any combination thereof

### Other Uses of the Humanized Mice

The humanized mice may be used to grow human T cells for immune therapy of patients, i.e. adoptive therapy.

The humanized mice may also be used for cloning T cell receptors (TCRs) that are protective against patient-specific cancers.

The humanized mice may also be used to grow human B cells for immune therapy and isolation of human IgG for immune therapy.

Another use for the humanized mice is for the growth of human immune regulatory cells for treatment of patients with immune disorders.

A further use is for determining rapid patient specific cancer therapy for multiple tumors.

The humanized mice may also be used to infect with human pathogens and to isolate protective T and B cells for treatment of humans.

The humanized mice may also be used as a vaccine and immune therapy model, for example as further described herein.

The humanized mice may also be used for: delivery of SCF, delivery of TPO, delivery of FLT3, deliver of c-Kit, delivery of CSF-1 or CSF-2, delivery of EPO, delivery of hTPO. In some embodiments, the delivery of the above is by plasmid to drive human immune populations for immune reconstitution.

The humanized mice may also be used for delivery of IL-15 to drive functional human T cell production.

The humanized mice may also be used for restoring immune function after chemotherapy and radiation, or for restoring immune function after bone marrow transplant, or for rebuilding the immune system in vivo.

These can all be for immune therapy and for treatment in the clinic.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Methods

### Tissues and Cell Lines

Human melanoma tissues were obtained in accordance with informed consent procedures approved by the Internal Review Boards of the Hospital of University of Pennsylvania and The Wistar Institute, Philadelphia. Fetal liver and thymus tissues were obtained from Advanced Bioscience Resources, Alameda, Calif. Human melanoma cell lines (A375, 451LU, WM3629) have been previously described (Fang D et al Cancer Res 2005, 65(20):9328-9337) and they were cultured in DMEM or RPM11640 medium supplemented with L-Glutamine and 5% FBS. All cell lines were tested for mycoplasma and short tandem repeat profile (DNA identity) before being used for any experiments.

### In Vivo Mouse Studies

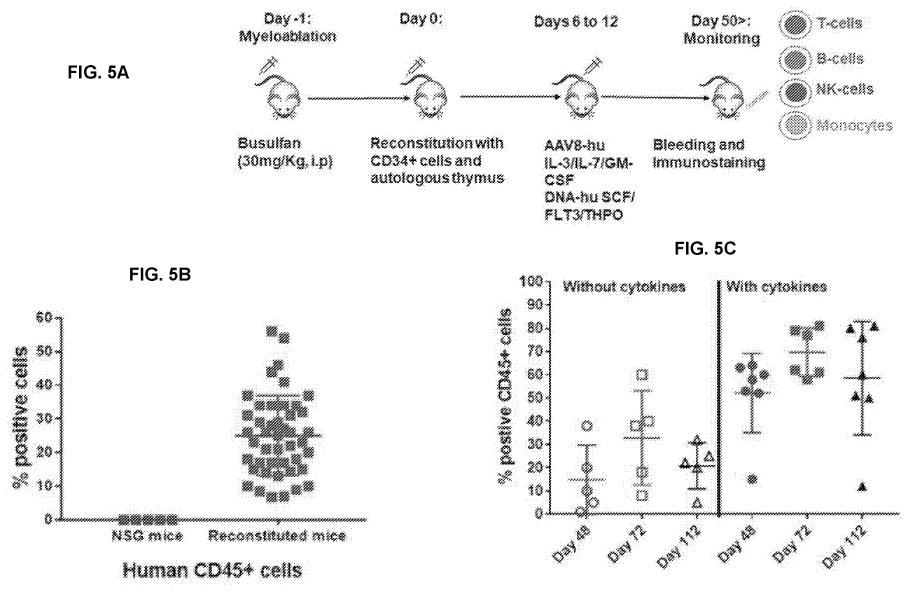

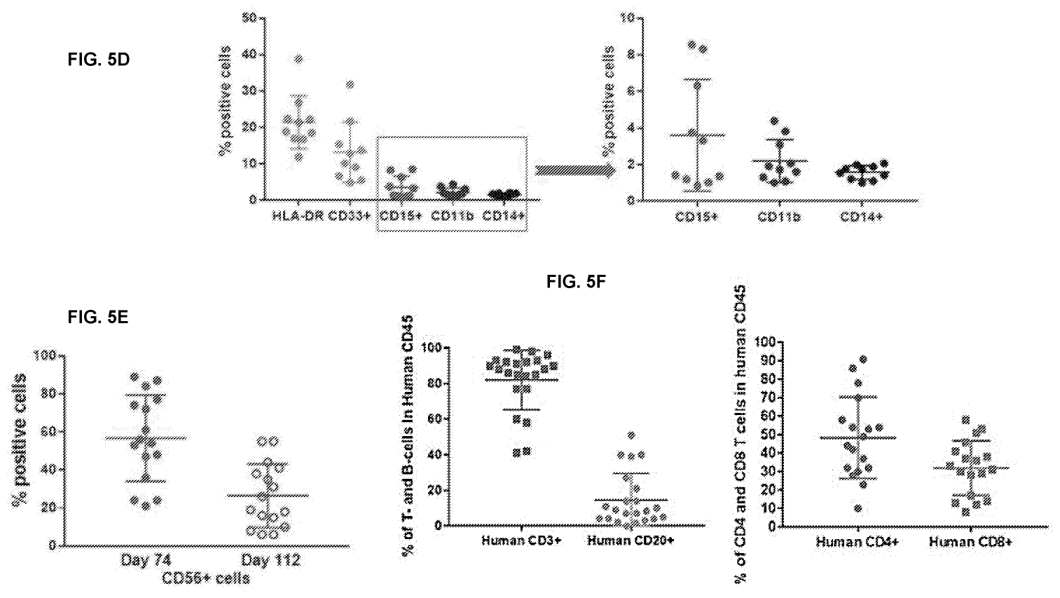

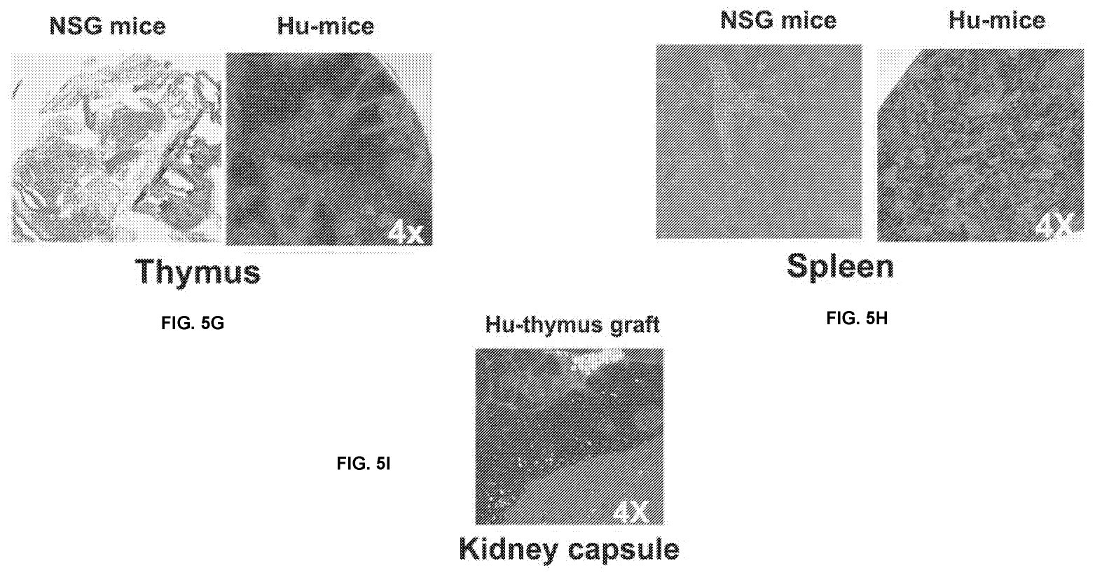

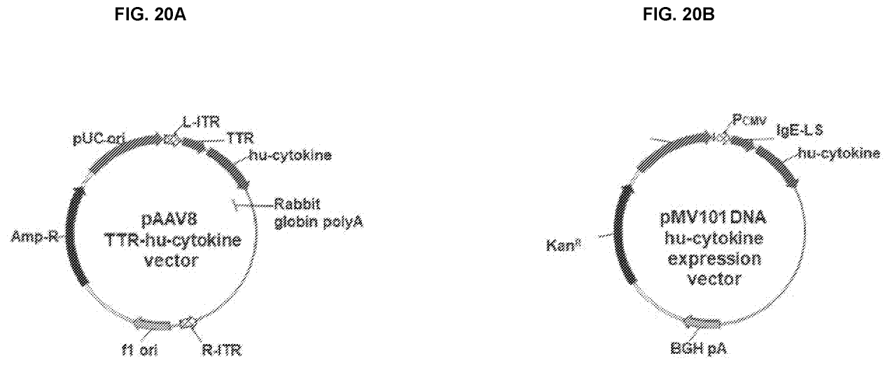

All animal experiments were performed according to protocols approved by the Wistar Institute's Institutional Animal Care and User Committee (IACUC). NOD/LtSscidIL2Rγ^{null} (NSG) mice were inbred at The Wistar Institute under license from the Jackson Laboratory. For humanization, fetal liver and thymus were obtained from the same donor (18-22 weeks of gestation). NSG mice (6 to 8 weeks) received thymus graft (1 mm³) in sub-renal capsule 24 h post myeloablation using Busulfan (30 mg/kg, i.p.; Sigma-Aldrich [B2635], St. Louis, Mo.). This is immediately followed by injection of autologous liver-derived CD34⁺ hematopoietic stem cells (10⁵ cells/mouse, i.v., FIG. 5A) that was magnetically sorted by microbeads conjugated with anti-human CD34 (Miltenyi Biotec., [130-046-703], Auburn, Calif.; Chen et al. Bio Protoc. (2013) 3(23)). Six to 8 weeks (>50 days) later, presence of human immune cells was monitored by multi-color flow cytometry using 18 color BD LSR II Analyzer (BD Biosciences; Somasundaram et al. Nat. Commun. 2017, 8(1):607). To accelerate human immune cell reconstitution mice received AAV8 encoding human IL-3, IL-7 and GM-CSF (FIG. 5; FIG. 20A; 10⁸; iv; Wu, . . . Ertl., et al 2013). Some groups for mice also received DNA plasmid delivery (electroporation of anterior tibialis muscle; 50-100 ug DNA) of constructs encoding FLT3, SCF, THPO (FIG. 5; FIG. 20B). Mice were considered humanized if human CD45 reached ˜25% in the peripheral blood of animals. All reconstituted mice were assigned into experimental groups according to the number of human immune cells (CD45+ and CD8+). Mice were subcutaneously injected with HLA-A allele melanoma cells (10⁵) over the right flank. All tumors were treated once they became palpable (˜100 mm³) with anti-PD-1 (10 mg/kg; 1× weekly; 5-6 injections; Keytruda, Merck, Rahway, N.J.) antibody and respective IgG antibody was used as control at similar dosage and frequency. Select groups of mice received Sunitinib (20 mg/kg, oral gavage, daily; Cancer Therapy Evaluation Program [CTEP], NCI, Bethesda, Md.) or a combination of Sunitinib and anti-PD1 antibody for 5-6 weeks. Hu-mice that showed complete regression of tumors were given a drug holiday of 4 weeks and then re-challenged with half the number of same tumor cells. Tumors were measured twice a week using digital calipers.

### HLA Typing

Fetal liver or melanoma cell genomic DNA was isolated using a GeneJET genomic DNA purification kit (Thermo Fisher, [K0722], Waltham, Mass.). Standard PCR was performed using the following HLA allele specific primers purchased from Integrated DNA Technologies (Coralville, Iowa): HLA-A1 forward 5′-ACA GAC TGA CCG AGC GAA (SEQ ID NO: 1) and reverse 5′-CTC CAG GTA GAC TCT CCG (SEQ ID NO: 2); HLA-A2 forward 5′-GAC GGG GAG ACA CGG AAA (SEQ ID NO: 3) and reverse 5′-CAA GAG CGC AGG TCC TCT (SEQ ID NO: 4); HLA-A3 forward 5′-CGG AAT GTG AAG GCC CAG (SEQ ID NO: 5) and reverse 5′-CAC TCC ACG CAC GTG CCA (SEQ ID NO: 6); HLA-A9 forward 5′-CAC TCC ATG AGG TAT TTC TC (SEQ ID NO: 7) and reverse 5′-CAA GAG CGC AGG TCC TCT (SEQ ID NO: 8); b2 micro-globulin forward 5′-CGA TAT TCC TCA GGT ACT (SEQ ID NO: 9) and reverse 5′-CAA CTT TCA GCA GCT TAC (SEQ ID NO: 10); b-actin forward 5′-TGC TAT CCC TGT ACG CCT CT (SEQ ID NO: 11) and reverse 5′-CCA TCT GCT CGA AGT CC (SEQ ID NO: 12). PCR cycling conditions were as follows: an initial denaturation step at 95° C. for 5 min and 30 cycles of denaturation (95° C., 30 s), annealing (56° C., 30s) and extension (72° C., 30 s) followed by a final extension at 72° C. for 10 min (Ref; Browning et al PNASe 1993)). The HLA A locus sequence was determined using the SeCore kits (One Lambda) and Applied Biosystems 3130×1 Genetic Analyzer (Thermo Fisher). HLA sequence analysis software (uType Dx) was used for analysis and allele assignment.

### Immunostaining

IHC staining was performed as previously described (Somasundaram et al 2017). Briefly, tissue sections were subjected to antigen retrieval by incubation with Target Retrieval Solution (Citrate [S1699] or Tris-EDTA buffers [S2367]; Agilent-DAKO, Santa Clara, Calif.) kit at 95° C. for 20 min and subsequently incubated with primary antibodies with optimum dilutions (FOXP3 [1:10]; CD4, HLA class I, HMB45 and mast cell tryptase [all at 1:100 dilution]; CD68, IgA and IgE [all at 1:400 dilution] and CD8 [1:500 dilution]. For detection of primary antibodies slides were incubated with anti-mouse, anti-rat or anti-rabbit antibodies at 1:1000 dilution and visualized by DAB (SK-4100) or AEC (SK-4200; both Vector Laboratories, Burlingame, Calif.) chromogens.

### Multiplexed Tissue MassCyTOF Staining

CyTOF staining was performed as previously described (Wang et al 2019). Briefly, carrier-free antibodies were commercially obtained and tagged with lanthanide metals using the Maxpar X8 metal conjugation kit from Fluidigm^{R} (201300, Ontario, Canada). Antigen retrieval was performed on deparaffinized tissue sections at 95° C. for 30 min in Tris/EDTA buffer, slides were cooled, blocked with 3% BSA-PBS solution and incubated with cocktail of antibodies (100 ul) overnight at 4° C. Next day slides were washed 3× with PBS and labeled with 1:400 dilution of Intercalator-Ir (Fluidigm 201192B) in PBS for 30 min at RT. Slides were washed with water (3×) and air dried for 30 mins before image mass cytometry acquisition using Fluidigm Hyperion Imaging System.

### RNA-seq and CIBERSORT

RNA was isolated from spleen and tumor tissues obtained from pre- and post-therapy (anti-PD1) mice using Zymo Direct-Zol RNA miniprep kit (Zymo Research, [R2073] Irvine, Calif.). RNAseq was done using ScriptSeq RNA-seq library preparation kit (Illumina, [BHMR1205], San Diego, Calif.). Quality control of RNA and DNA library was done using the Tapestation 4200 and Bioanalyzer 2100 system (Agilent, Santa Clara, Calif.). Library quantification was done using the Kapa Library Quantification qPCR kit (Roche, [KK4854], Pleasanton, Calif.) and subjected to a 75 bp paired-end sequencing run on Illumina's NExtSeq 500 high output kit following the manufacturer's protocol. RNA-seq analysis was performed using RSEM v1.2.12 software and downstream expression analysis was done using Differential2 (Shuai Wu et al. Nature Commun. 2018; 9: 4166). RNA-seq data was used to enumerate tumor-infiltrating leukocytes using CIBERSORT, an analytical tool available online (cibersort.stanford.edu) (Chen et al. Methods Mol. Biol. 2018; 1711:243-259).

### Example 1-In Vitro Expression of Cytokines

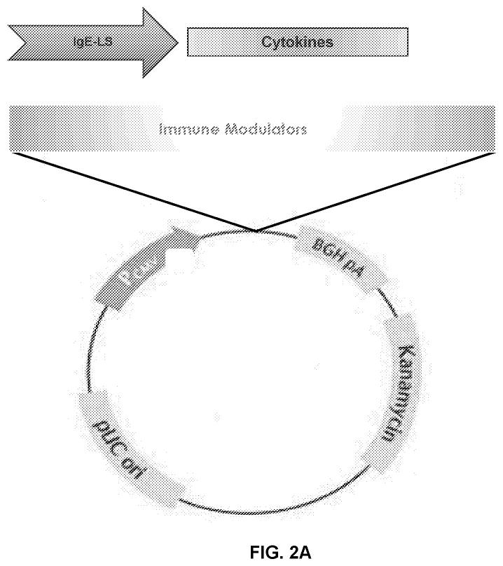

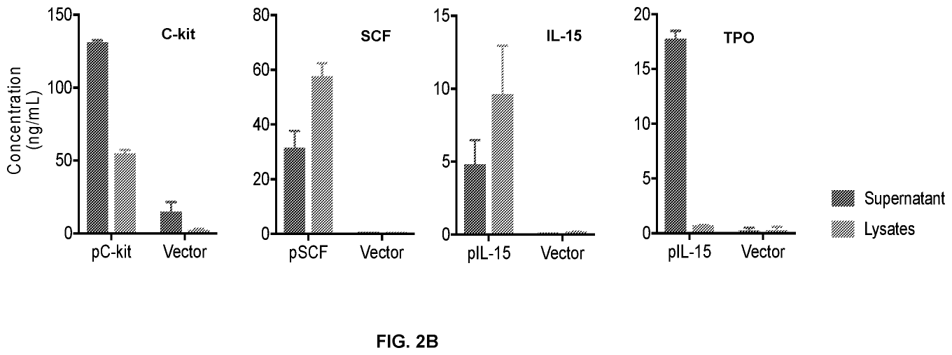

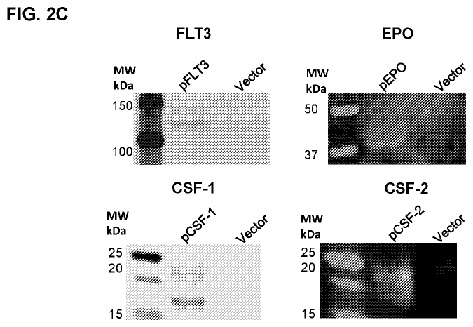

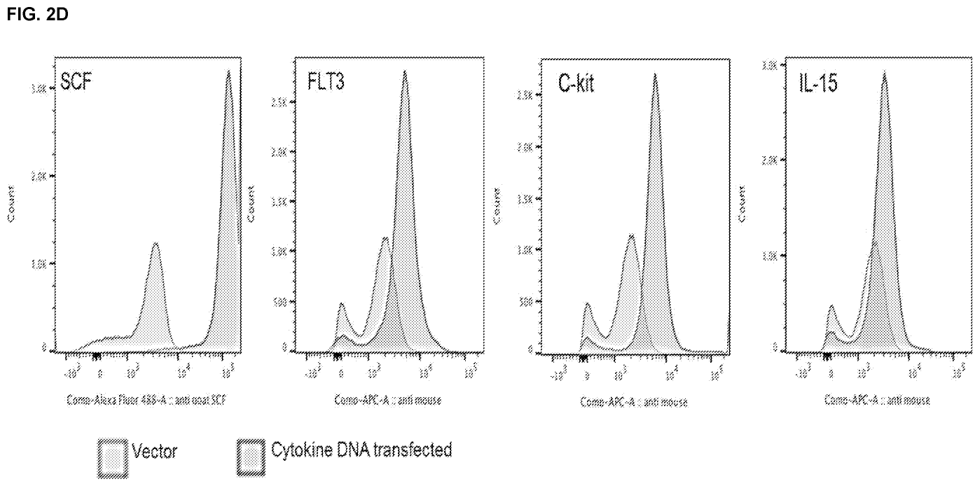

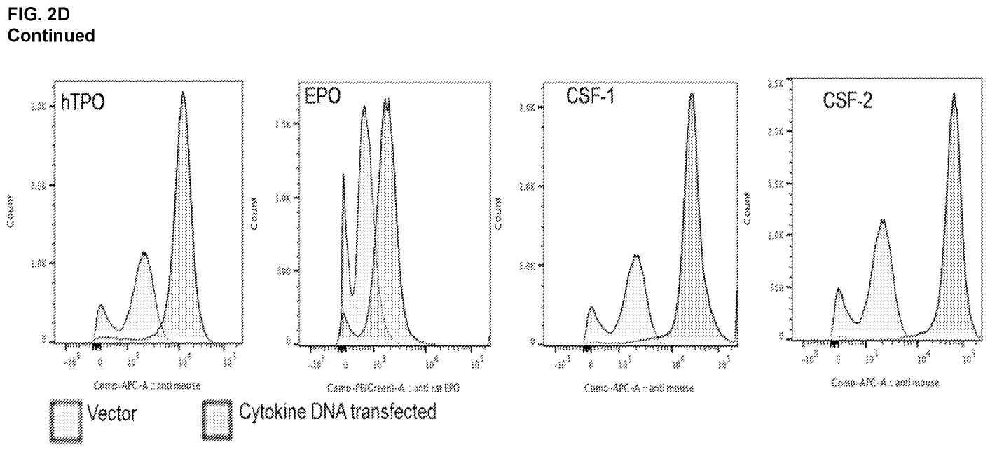

Various immune cytokine constructs were cloned into the mammalian expression vector pMV101. Expression of immune cytokines from the vector was verified by ELISA of 293T cells transfected with plasmids expressing immune cytokines. The levels of cytokines in transfected cells were analyzed by ELISA (FIG. 2A). Western blot analysis of transfected cells with respective antibody was conducted (FIG. 2C). Flow cytometry analysis of cytokines from the transfected cells was also conducted. For the flow cytometry analysis, two days post transfection with respective cytokines plasmids, transfected cells were stained with specific IgG (1:100) and then stained with the appropriate secondary conjugated IgGs. The cells were subsequently gated for FACS analysis as singlet and live cells (FIG. 2B). The percent of positive cells was indicated in histograms as indicated in FIG. 2B.

### Example 2—Time Course of Cytokine Expression

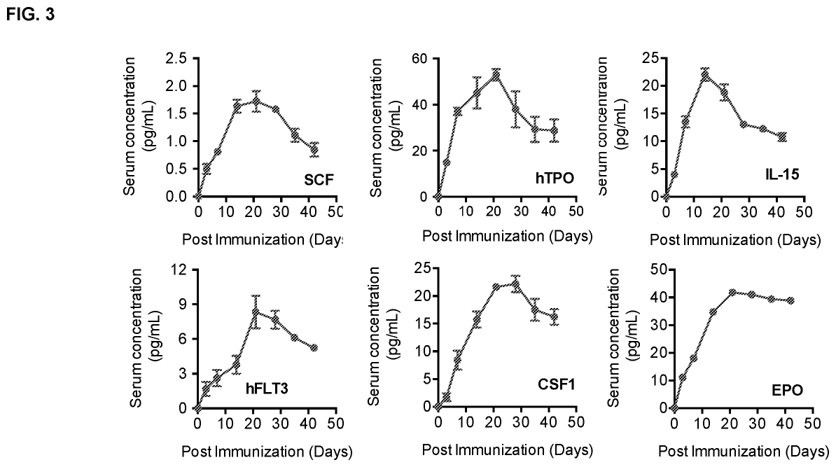

The concentration of immune cytokines was analyzed at various time periods from hu-Mice mice immunized with immune cytokines, and cytokine levels were measured by ELISA. Results shown in FIG. 3 are the means±SEMs of 2 to 3 mice per cytokines analyzed in duplicate.

### Example 3—Circulating Human Immune Cells in Humanized Mouse

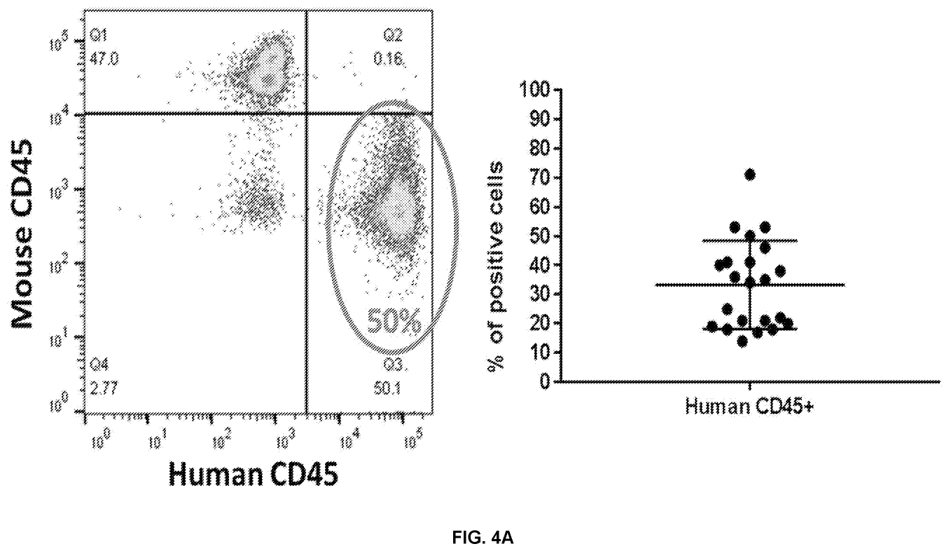

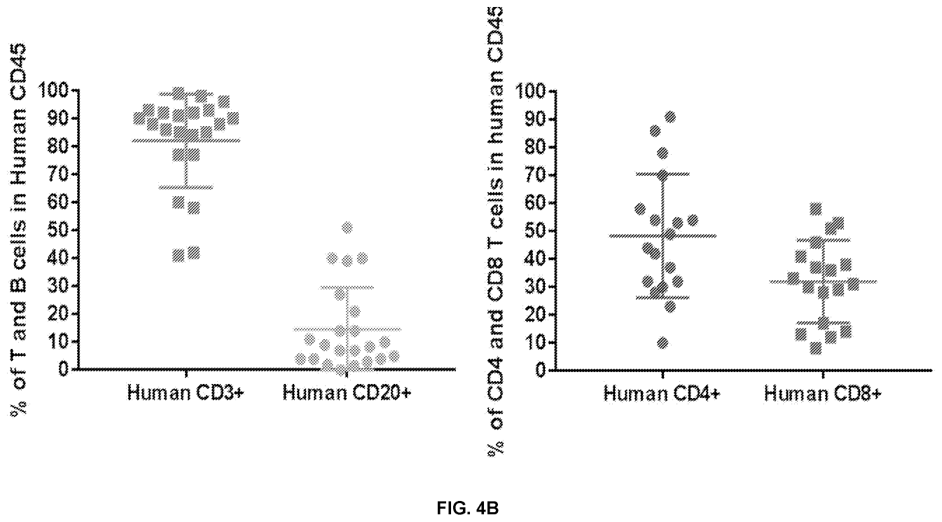

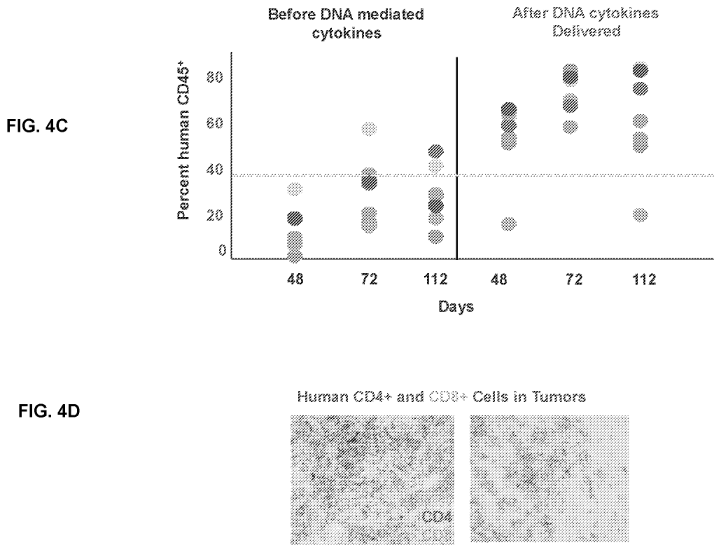

After 8-12 weeks, 20-50% of human CD45+ cells were observed in circulating blood in the mice. (FIG. 4A). Physiological levels of T- and B-cells are shown in FIG. 4B, left panel, and a normal human ratio of CD4/CD8 (2.0) is seen in FIG. 4B, right panel. Improved reconstitution of hu-Mice with human lymphocytes populations after modified novel synthetic plasmid immune cytokines delivery is shown in FIG. 4C. A higher human CD45 population was generated (FIG. 4D).

### Example 4—Cellular Immunity in huMice

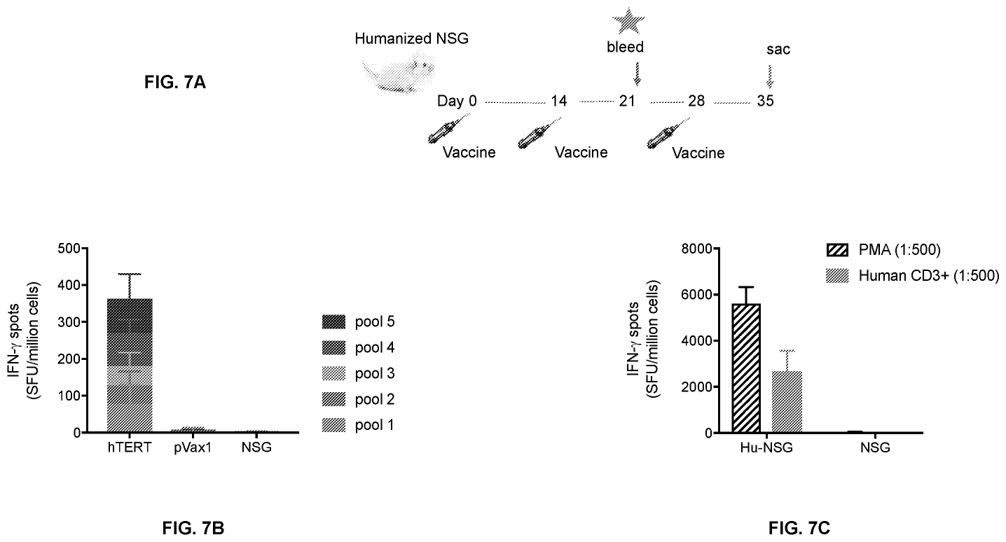

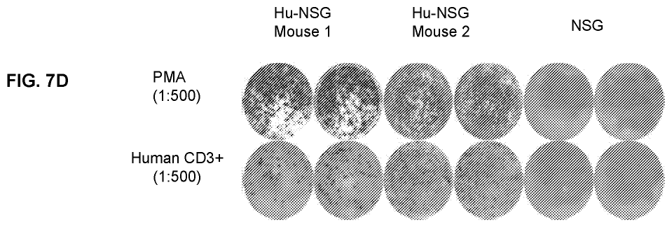

The timeline of DNA immunizations and immune analysis used in the study is shown in FIG. 7A. NSG-humanized mice were immunized three times, each 2 weeks apart, with 25 μg of pVaxl vector or human TERT plasmid and sacrificed 1 week after the 3rd immunization. Splenocytes harvested 7 days after the third immunization were incubated with pools of individual human TERT peptides (15-mers overlapping by 11 amino acids) as shown in FIG. 7B. PMA or anti-CD3 stimulation and results are shown in stacked bar graphs in FIG. 7C. Data represent the average numbers of SFUs per million splenocytes from 4 mice/group with values representing the mean responses in each±SEM. Experiments were performed independently at least two times with similar results. FIG. 7D shows a representative ELISpot image from one sample for antigen.

### Example 5—Humoral Response Against Immune Antigen

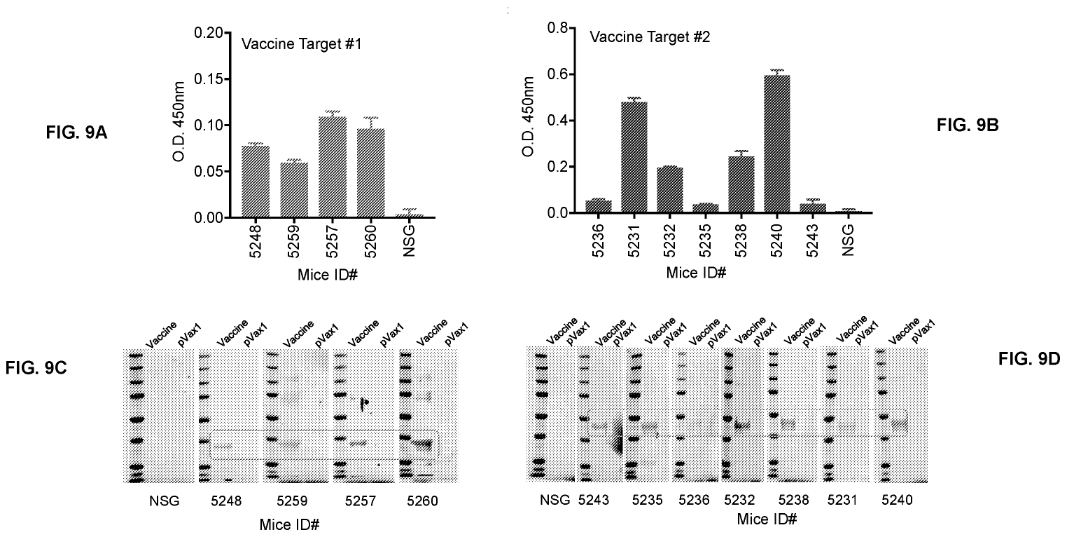

Groups of hu-Mice mice were injected with vaccine targets, and serum was collected at one week after the second immunization. Individual sera were assessed for antigen-specific IgG content by ELISA analyses. The results are shown in FIG. 9A and FIG. 9C. Each bar represents the serum value for an individual animal. Target vaccine was transfected in 293T cells and were lysed 48 hours post transfection and subjected to Western blot using immune sera that were raised in mice. Blocking was conducted overnight at 4° C. followed by 2 hours at room temperature (1:100 dilution) with primary antibody incubation. Both membranes were finally incubated in 1:5000 secondary antibody (Goat anti-Human IgG) for 1 hour. The blots were then washed and the membranes were imaged on the Odyssey infrared imager (LI-COR). Lane 1 contains the protein molecular weight markers (kDa). The results are shown in FIG. 9B and FIG. 9D.

### Example 6—Induction of Human IgA+in Hu-NSG Mice+Cytokine Delivery

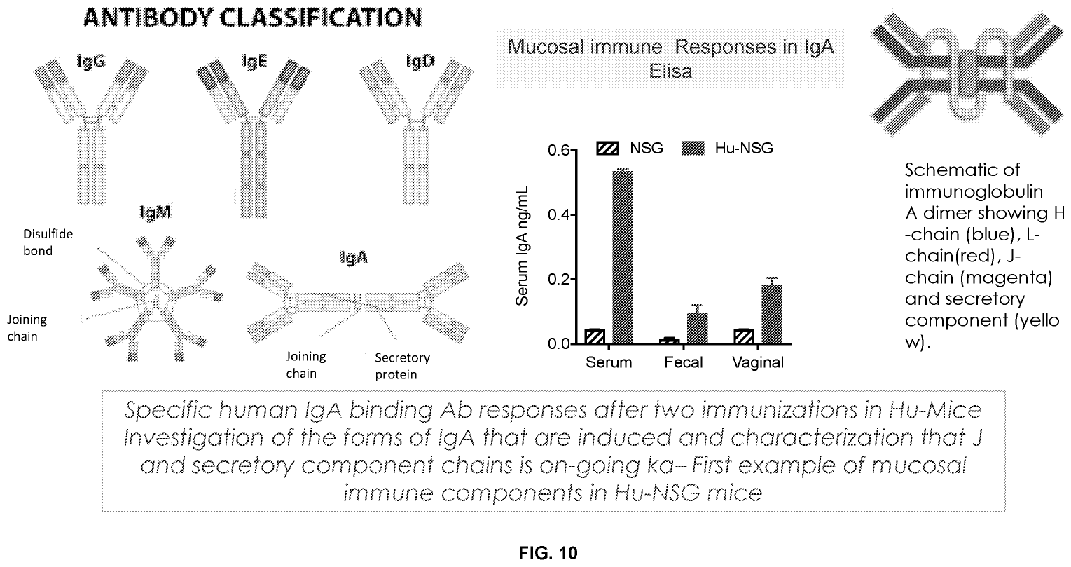

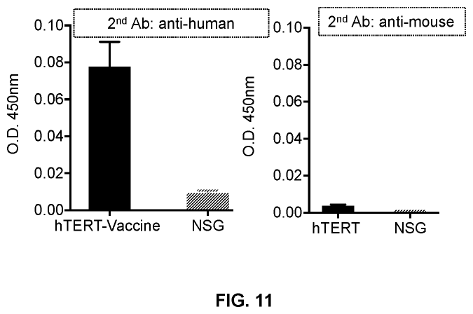

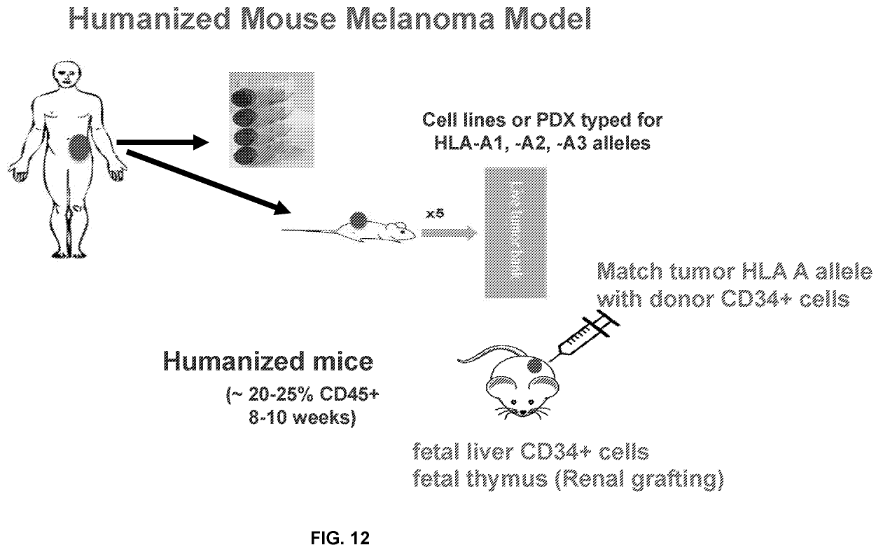

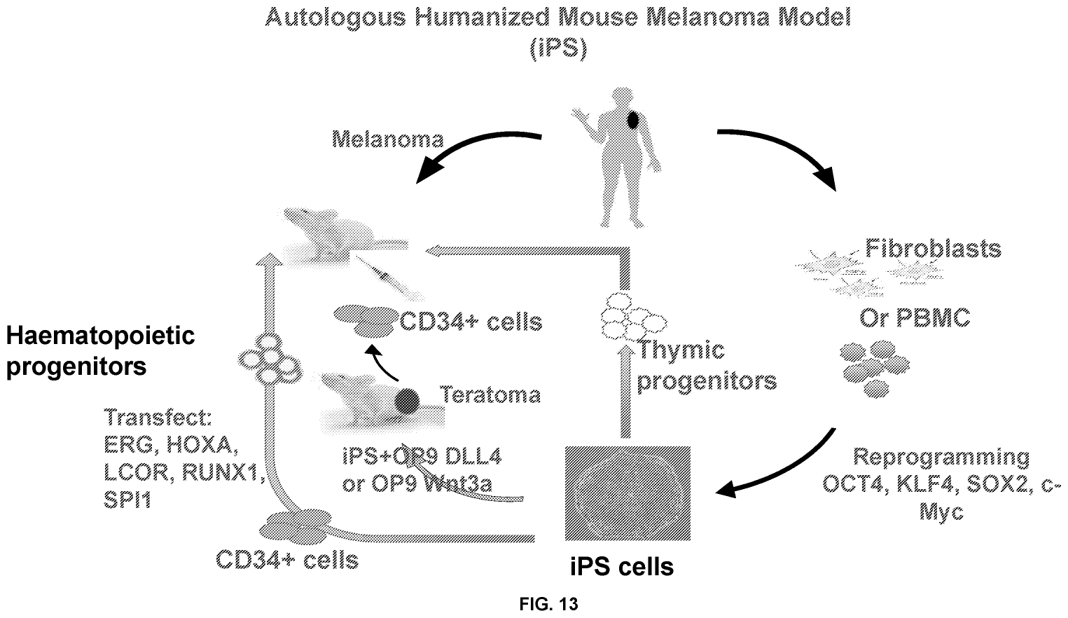

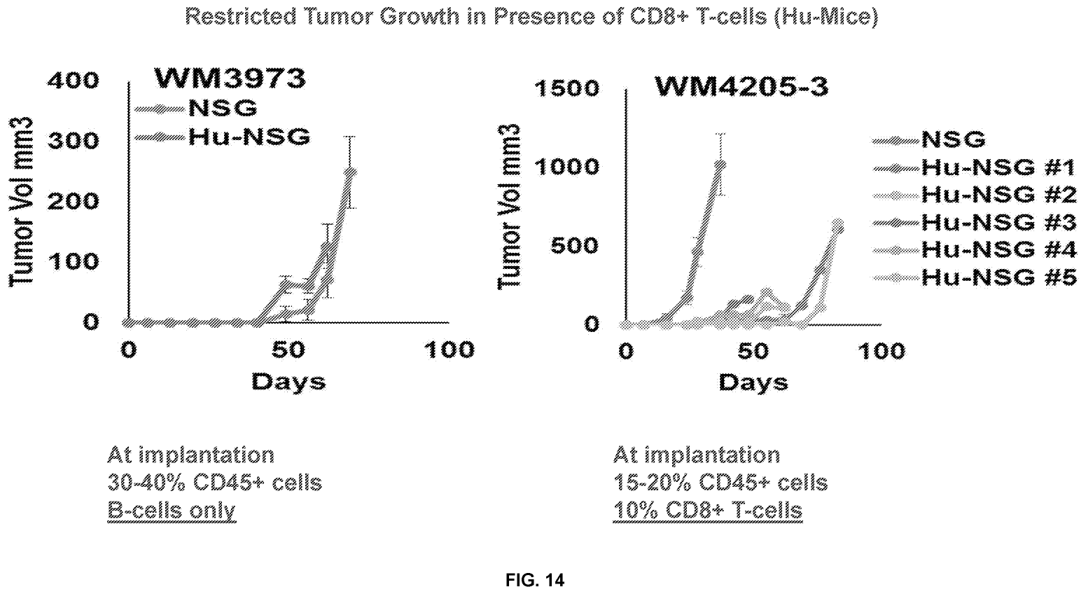

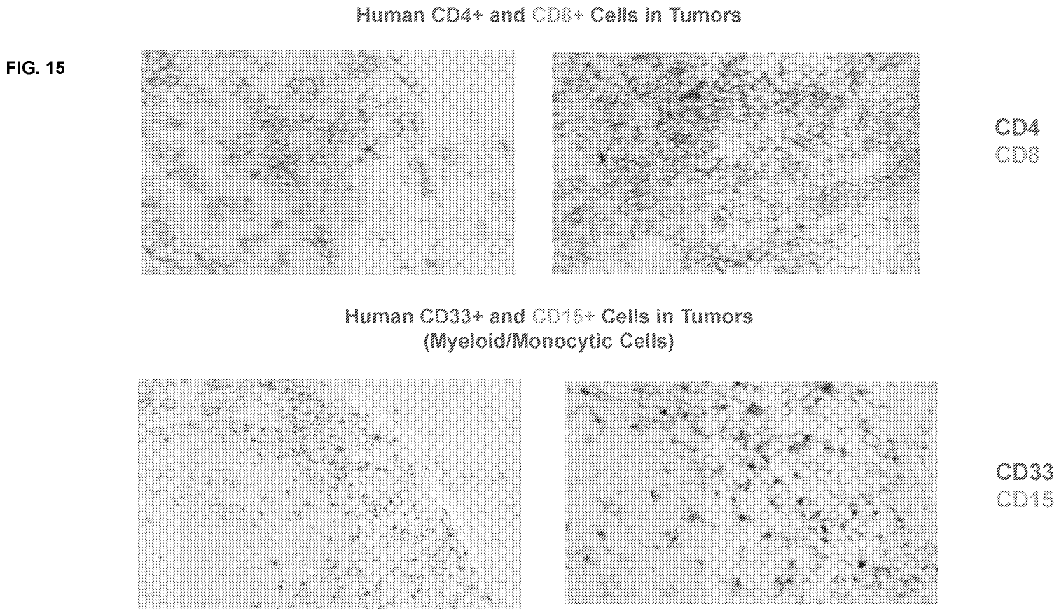

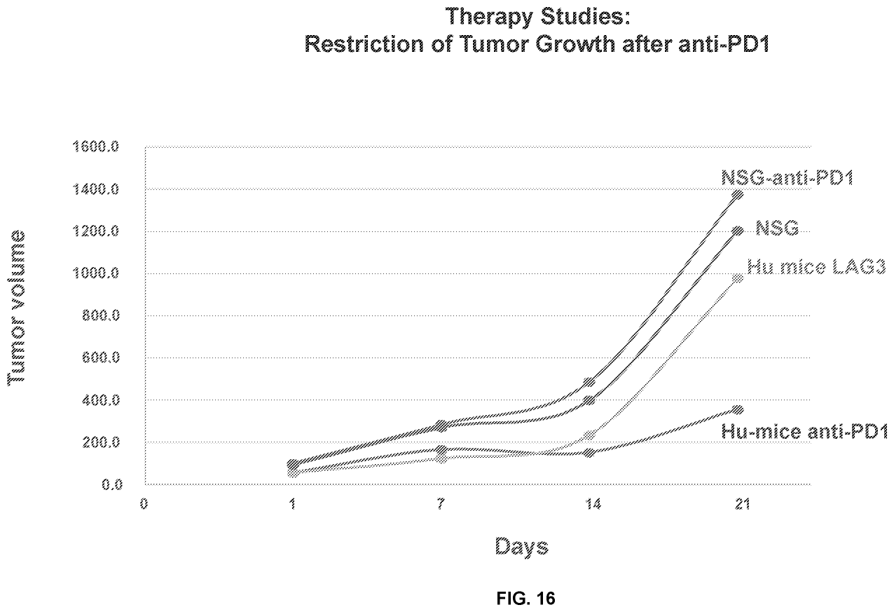

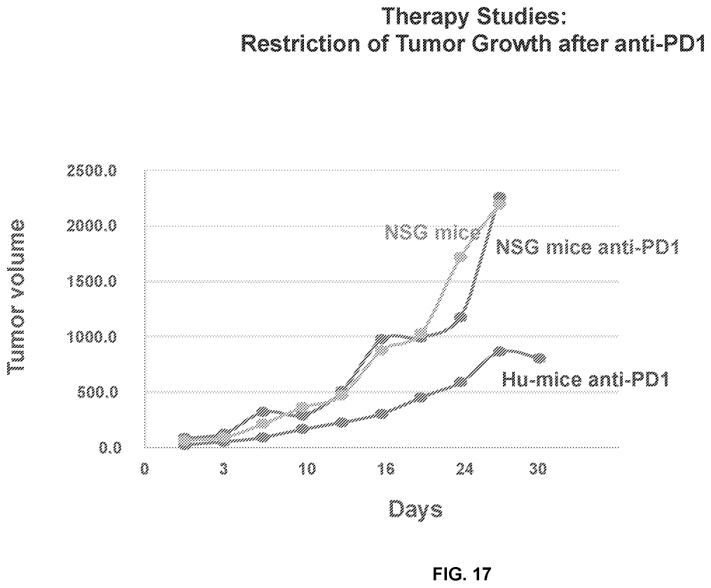

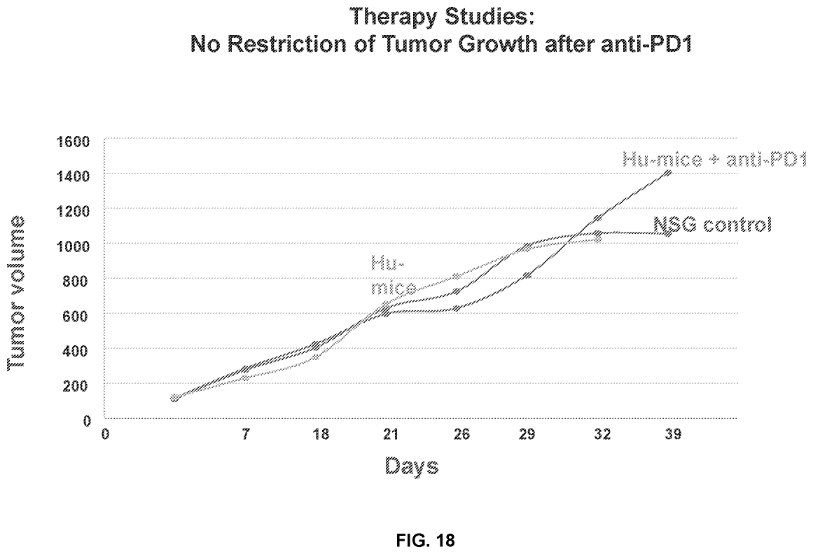

IgA is a hallmark of mucosal B cell immunity. The specific serum IgA anti-vaccine antibodies as indicated in mice that received the targeted immunization and were assayed by ELISA. The results are shown in FIG. 10. Standard errors are as shown. FIG. 10 shows specific human IgA binding Ab responses after two immunizations in Hu-Mice. This is the first example of mucosal immune components in Hu-NSG mice.

### Example 7-Induction of Human Antibodies in Hu-NSG Mice Against Human Tumor Antigen

ELISA plates were coated with hTERT transfected 293T cell lysates and primary antibody were used from immune sera from hTERT vaccinated (1:50) and then 2nd Ab: either anti human IgG-HRP (left) (1:10000) or anti mouse IgG-HRP (right) (1:6000) and measured by ELISA analysis. OD, optical density. The results show seroconversion and specificity of human responses in Hu-Mice. These mice should serve as a rich resource for novel reagent production.

### Example 8-Tumor-Infiltrating Mast Cells Induce Therapy to Anti-PD1

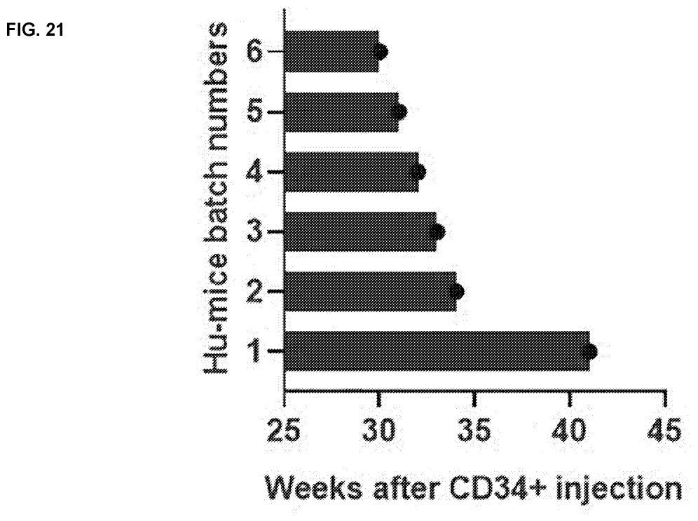

An advanced Hu-mouse model was used in this study to delineate the mechanism of immune resistance to anti-PD1 therapy. In contrast to transgenic humanized mouse chimeras that produce growth and differentiation factors continuously, in the present model a targeted and sequential delivery of cytokine factors is provided by transgenes encoded in AAV8 or pMV101 DNA-based vectors (see FIG. 20) to promote human immune cell reconstitution. Unlike other available Hu-mice, the present model provides a stable life span of approx. 30 weeks (FIG. 21) after human CD34+ cell injections. The long-term stability of the present model offers an opportunity to characterize treatment responses to immune-based therapies after human tumor challenge.

In the Hu-mouse of the invention. 8 to 12 weeks after CD34+ cell injection, a robust number of human CD45+lymphocytes were observed in the peripheral blood when compared to circulating blood of non-reconstituted NSG mice (FIG. 5B). Delivery of AAV8 hu-cytokines (IL-3, IL-7 and GM-CSF) significantly increased the number of human CD45+ cells in mouse peripheral blood circulation when compared to the group that did not receive any cytokines (FIG. 5C). Addition of cytokines such as SCF, FLT3 and THPO helps in T-cell and myeloid cell differentiation but does not enhance the level of human CD45+ cells.

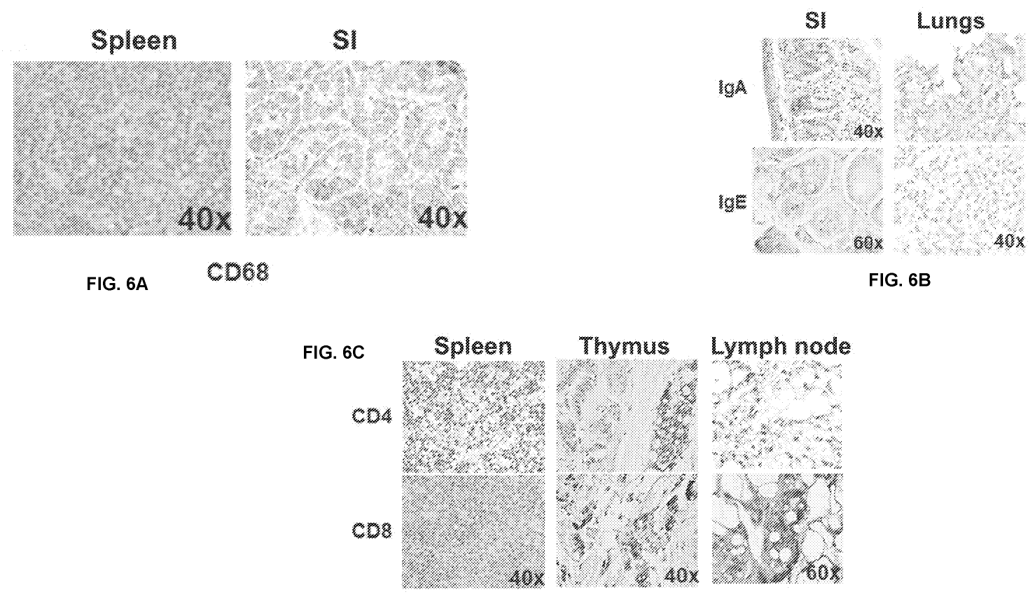

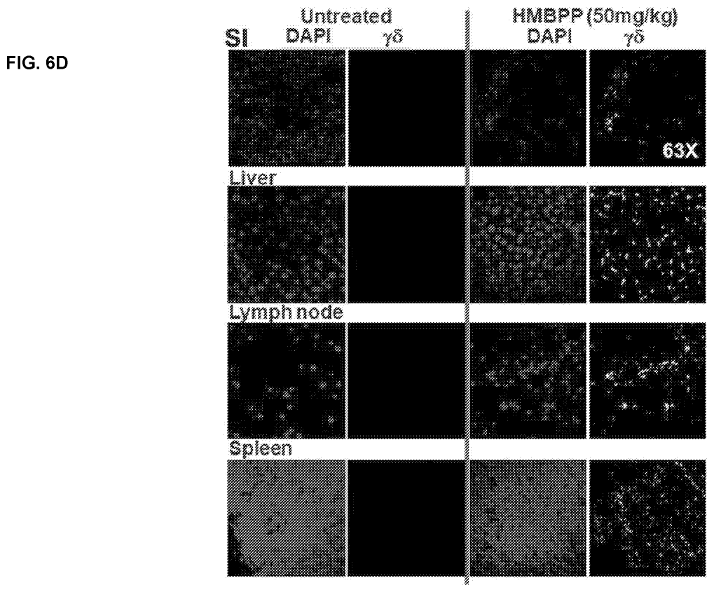

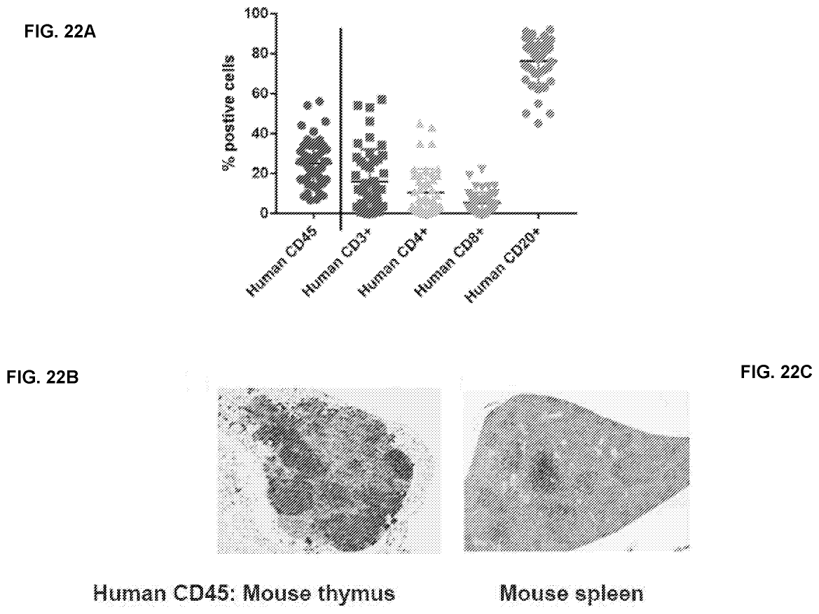

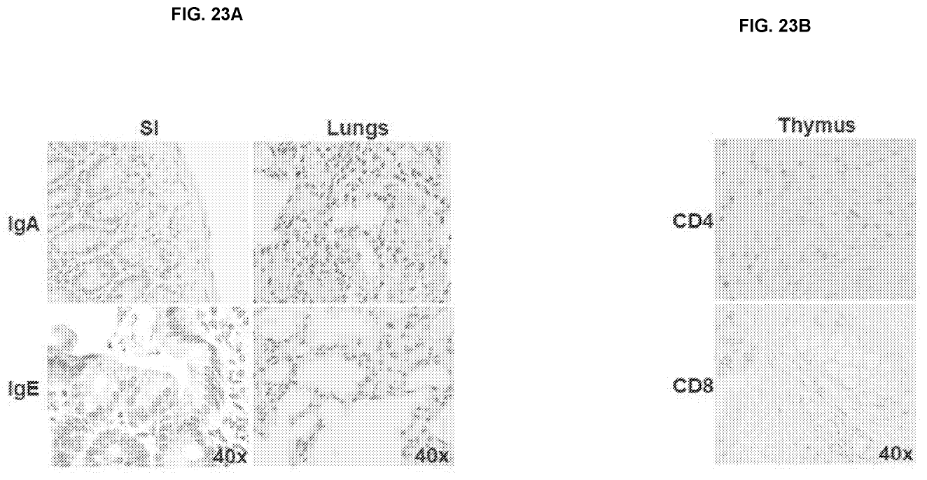

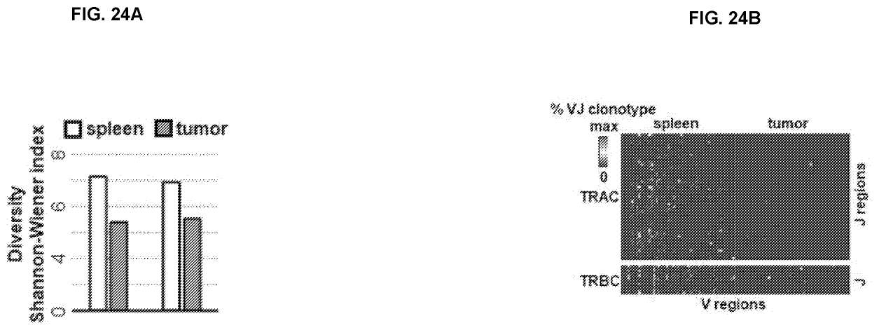

Besides the presence of human CD45+ cells, the presence of monocytes/myeloid lineage cells HLA DR+, CD33+, CD15+, CD11b+ and CD14+ was observed (FIG. 5D). NK-cells (CD 56+) (FIG. 5E), T-cells (CD3+, CD4+ and CD8+) and B-cells (CD20+) were also observed (FIG. 5F). NK-cell (FIG. 6E) and B-cell subpopulations (FIG. 22A) were initially high, but three to four weeks later their levels dropped down as the mouse thymus (FIG. 5G; FIG. 22B) mouse spleen (FIG. 5H, FIG. 22C) and the renal capsule-grafted hu-thymus (FIG. 5I) were repopulated with human lymphoid precursor cells that undergo differentiation. Immunodeficient NSG mice, due to their IL2Rγ^{null} genotype, have underdeveloped lymph nodes and hence it was impossible to obtain enough tissue material for characterization of this lymphoid organ. Efficient antigen presentation to T- and B-cells depends on macrophages (CD68+) and they were observed in the spleen and small intestine (FIG. 6A). B-cells are fully functional, as antigen specific IgG were detected in circulating blood (see anti-hTERT response below), and IgA and IgE with unknown specificity in the mucosal layers of small intestines and the lungs (FIG. 6B, FIG. 23). Human CD4+ and CD8+ subpopulations of T-cells were detected in spleen, thymus and mesenteric lymph node tissues (FIG. 6C). Most T-cells in the lymphoid organs have diverse expression of TcR α/β+ chains (FIG. 24) and a frequent presence of tissue resident T-cells in the liver, mesenteric lymph nodes and in the spleen are TcR γ/β+. These cells are further expanded in the presence of hydroxy-2-methyl-2-butenyl 4-pyrophosphate (HMBPP), a bacterial metabolite, that specifically activates human γ/δ+ T-cells (FIG. 6D). T-cells expressing TcR γ/δ+ chains are known to protect against pathogens in mucosal or epithelial layers; as their functional activity is HLA unrestricted, their potential use in adoptive T-cell therapy is being explored in solid tumors.

### Example 9—Hu-Mice Were Immunized with hTERT DNA Vaccine

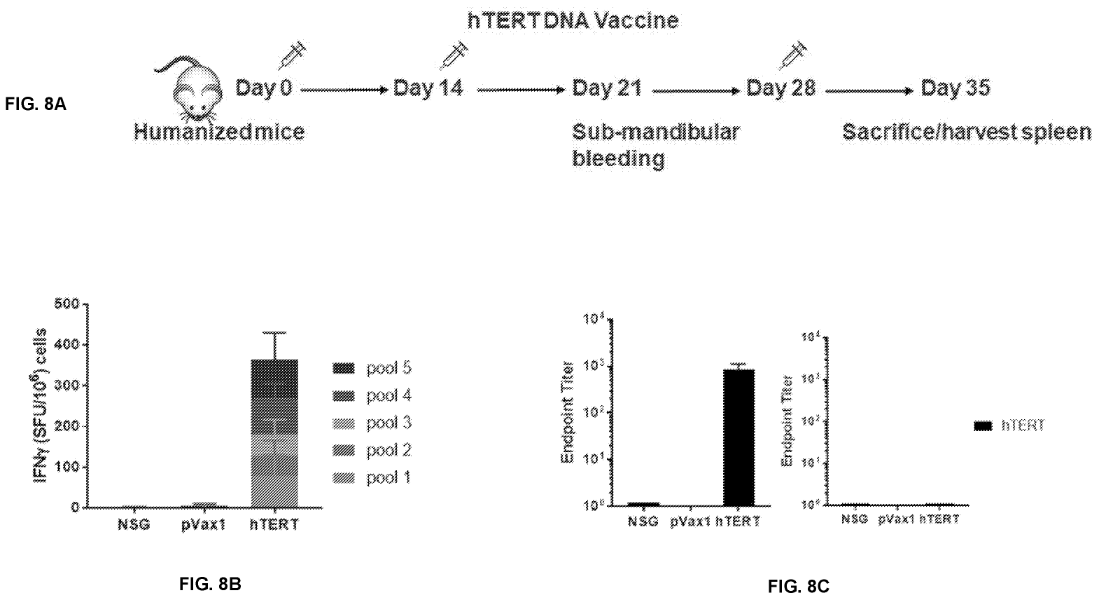

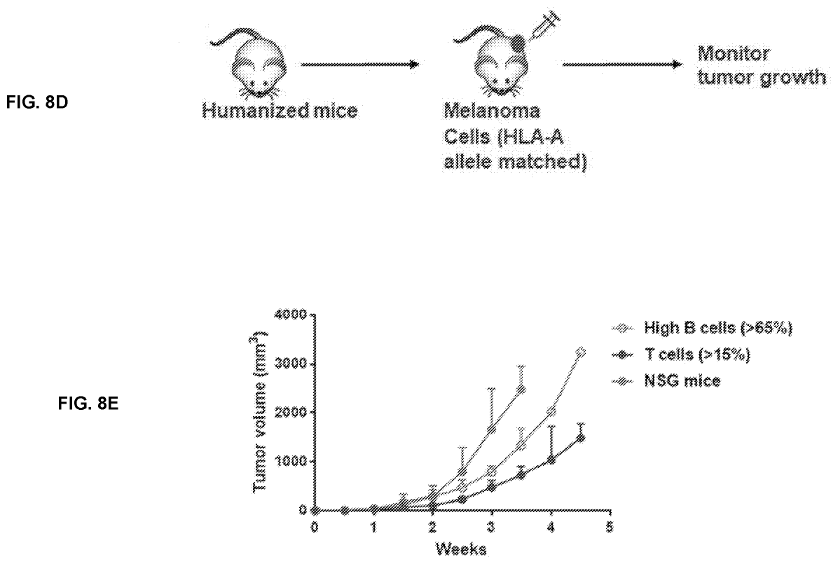

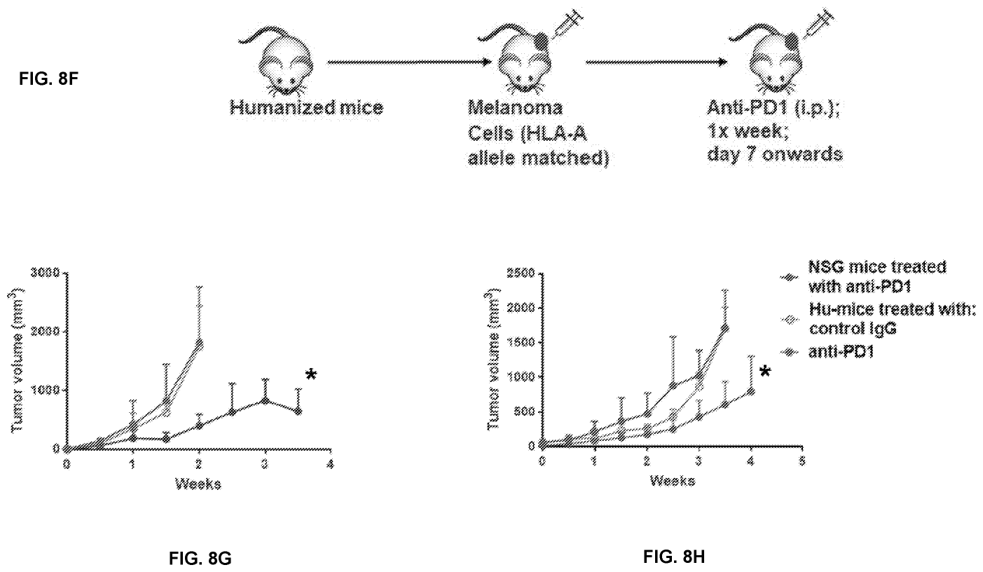

If mice are fully reconstituted with human lymphoid cells, then it is necessary to determine the functionality of the humoral (B-) and cellular (T-) immune cell compartment and their ability to respond to an immunizing agent that frequently requires antigen presentation to T- and B-cells. For this, Hu-mice were immunized with hTERT DNA vaccine, a universal tumor-associated antigen (FIG. 8A), and the lymphoid cells in the spleen were tested for their ability to respond to hTERT antigen after in vitro stimulation followed by IFNγ ELISPOT assay. Anti-TERT specific T-cell responses were observed on a panel of overlapping peptides spanning the hTERT protein (FIG. 8B). In addition, the sera from hTERT immunized Hu-mice showed the presence of hTERT specific IgG antibodies confirming the functionality of B-cells (FIG. 8C). There were no T- or B-cell responses to pVax1 vector alone and the spleen cells from control non-reconstituted NSG mice did not respond to hTERT DNA vaccination (FIGS. 8B and 8C).

Next, it was determined whether T-cells have an ability to restrict tumor growth in the humanized melanoma mouse model (FIG. 8D). For this, Hu-mice with ˜15% circulating CD8+ cells in peripheral blood were challenged with melanoma cells that are HLA-A allele matched to donor CD34+ cells. Under these conditions there was a significant restriction of tumor growth when compared to non-reconstituted NSG mice or Hu-mice with high circulating B-cells (>65% CD20+; FIG. 8E) and negligible (<1%) CD8+ T-cells.

### Example 10—Anti-PD1 Therapy in Hu-Mice Melanoma Model

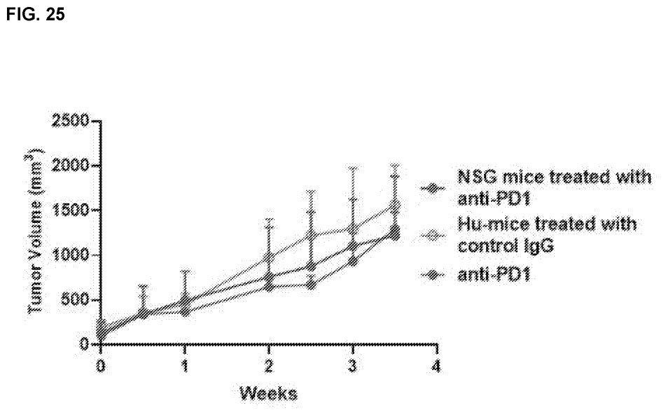

Next, if T-cells have an ability to restrict tumor growth, the question was addressed as to whether treatment of tumor bearing Hu-mice will benefit from anti-PD1 therapy. In an established tumor model (FIG. 8F), treatment with anti-PD1 antibody significantly restricted tumor growth of 2 different metastatic melanomas (WM3629 [HLA-A3; FIG. 8G] and A375 [HLA-A2; FIG. 8H]) when compared to tumor growth in Hu-mice treated with control IgG or non-reconstituted NSG mice treated with anti-PD1 antibody. In one other case of metastatic melanoma (451Lu) that is aggressively growing in Hu-mice, treatment with anti-PD1 had a negligible effect on growth (FIG. 25). No immune infiltrating cells were detected by IHC staining. Without wishing to be bound by theory, tumor burden is a limiting factor to anti-PD1 therapy responses in patients. Similar to patient responses, the present results in Hu-mice also demonstrate heterogeneous responses to antibody therapy.

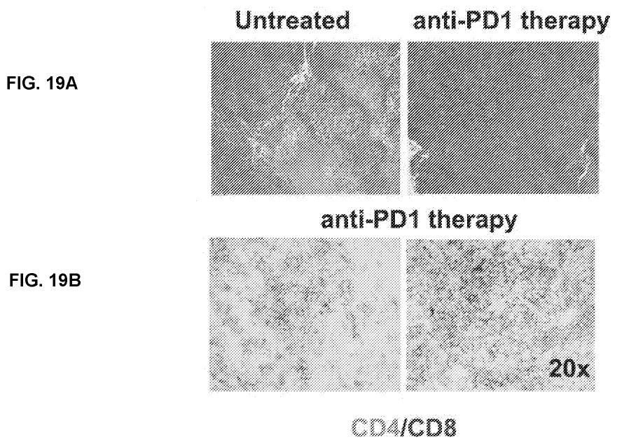

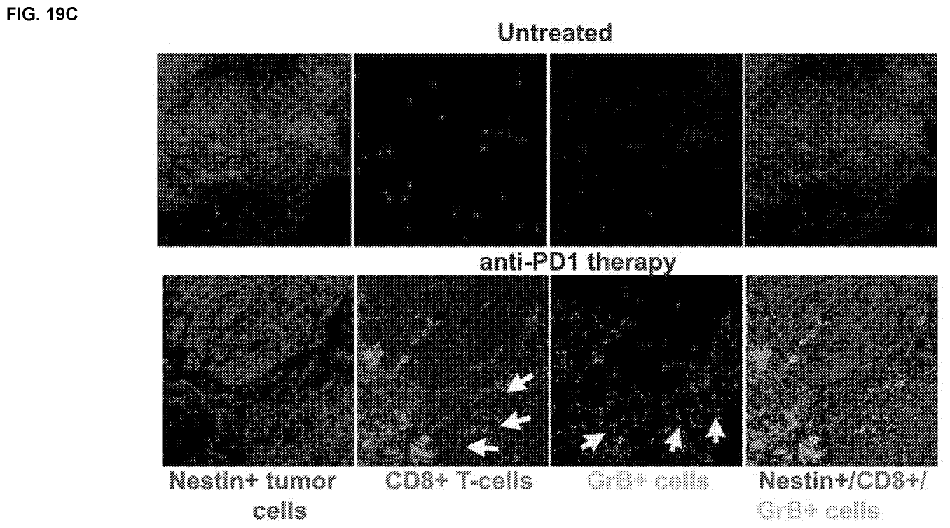

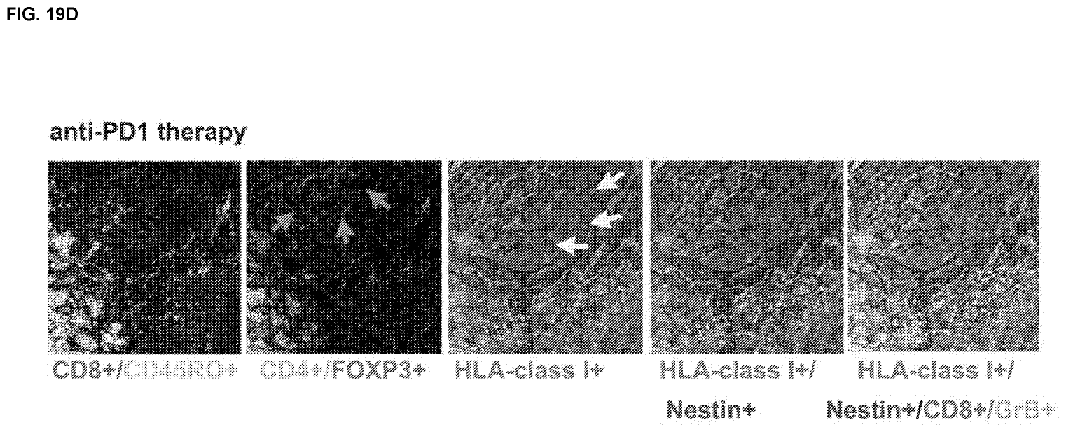

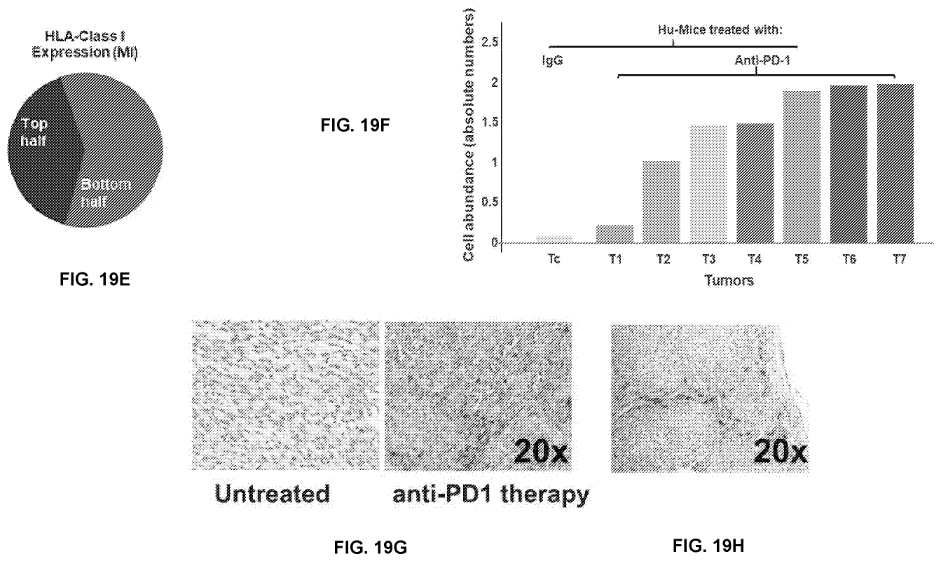

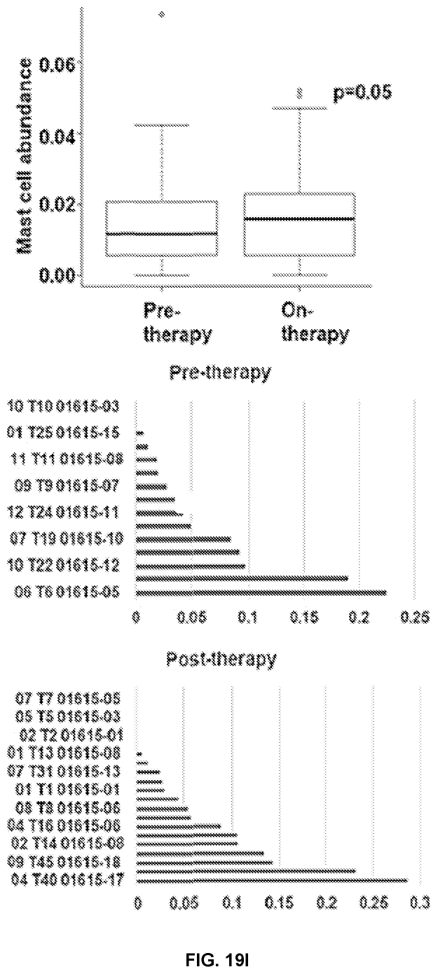

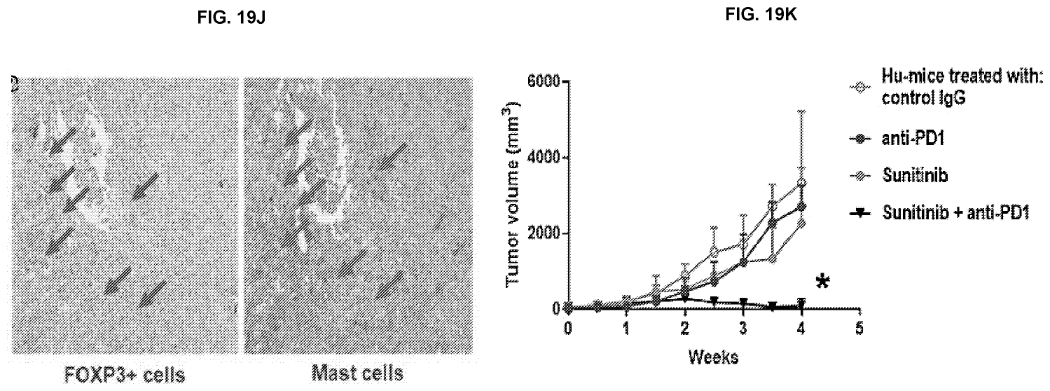

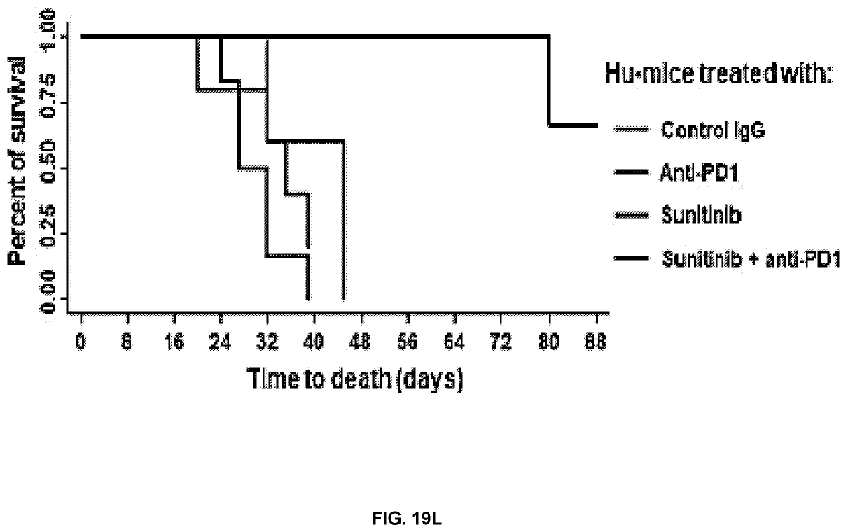

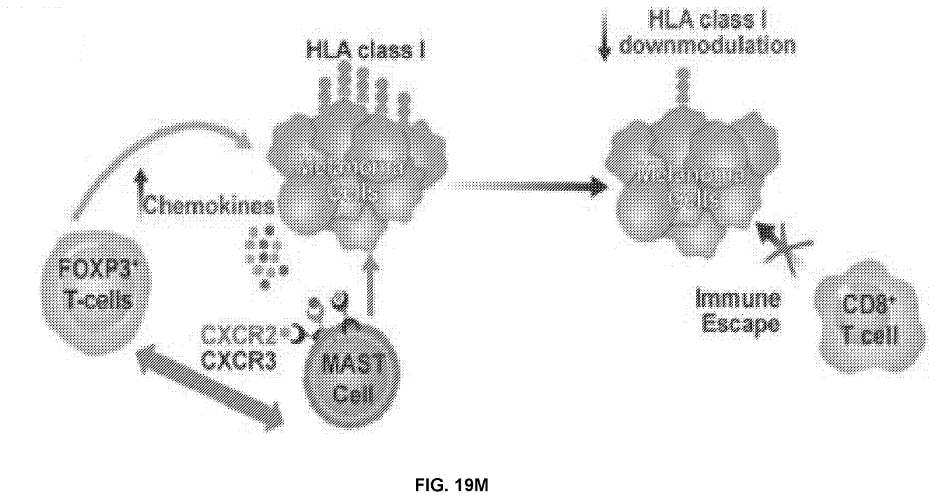

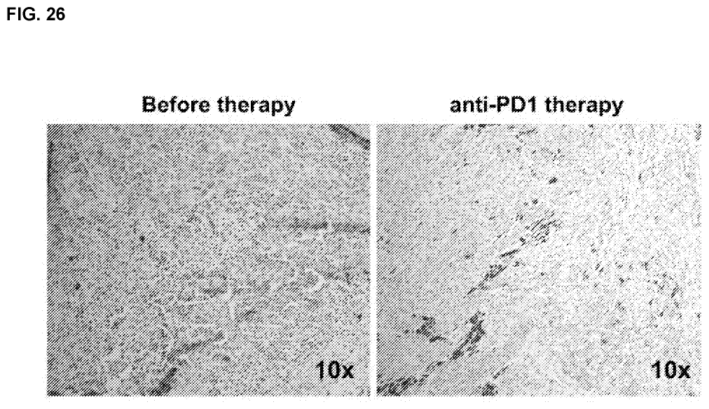

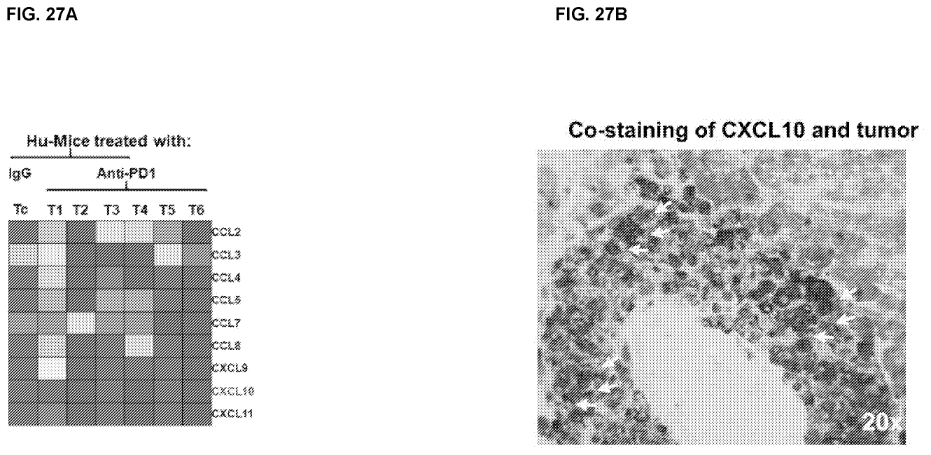

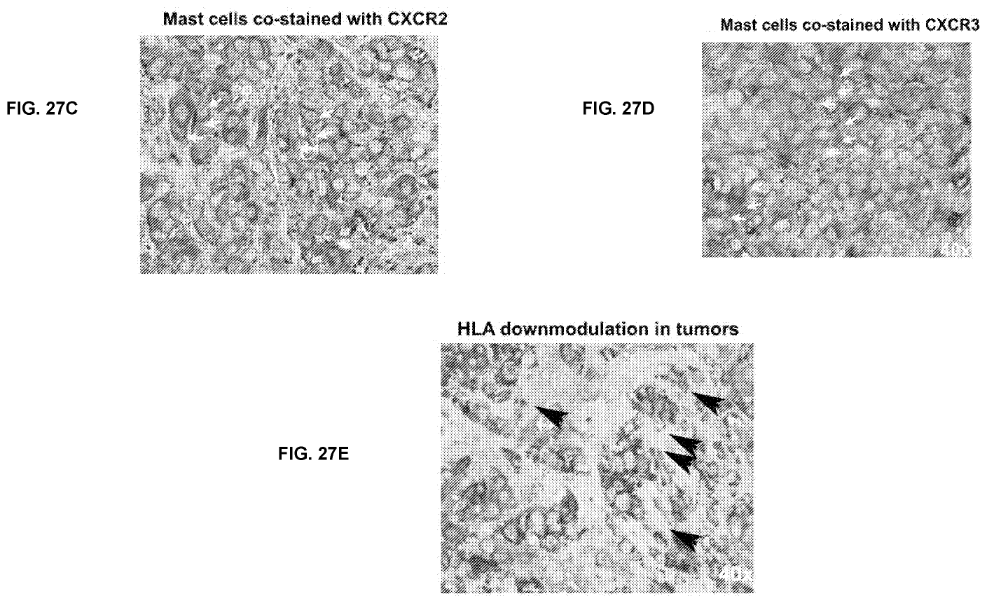

In order to understand the phenomenon of mixed therapy responses to anti-PD1 treatment, IHC, multiplexed imaging using MassCyTOF and RNA-seq was performed on tumors obtained from anti-PD1 treated Hu-mice and they were compared with control Ig treated mice. Higher levels of immune infiltration were observed in tumor sections of anti-PD1 treated Hu-mice when compared to untreated controls (see IHC staining; FIG. 19A). In addition, there was a heterogeneous distribution of CD4+ and CD8+ T-cells within the tumors of mice that received anti-PD1 therapy (FIG. 19B). Some regions of the tumors revealed poor infiltration of CD8+ T cells and this may have given rise to a therapy resistant tumor that continued to show unrestricted growth. Multiplex imaging of tumor tissue sections by MassCyTOF with a panel of 25 rare earth metal-tagged antibodies revealed selective distribution of CD8+/Granzyme (Gr) B+ T-cells (FIG. 19C, bottom 2 right panels) that were of an effector memory phenotype (CD45RO+; FIG. 19D [left most panel]) in mice that received anti-PD1 treatment whereas there was minimal infiltration of these cells in untreated mice (FIG. 19C, top panel). Further, there was an increased presence of FOXP3+ T-reg cells in areas that lacked CD8+ T-cell infiltration (FIG. 19D [2^{nd} panel from left]) and the same areas also had significant downmodulation of HLA class I expression (FIG. 19D [3^{rd} panel from left]) and FIG. 19E). To further study the mechanism of selective downmodulation of HLA class I expression, RNA-seq analysis of tumors from Hu-mice treated with and without anti-PD1 antibody was performed. To determine the immune phenotypes within the tumor cells, CIBER sort analysis of the RNA-seq data was performed and it revealed higher presence of tumor resident mast cells (see heat map, FIG. 19F). Immune histology staining of mast cells confirmed increased numbers in Hu-mice tumors that received anti-PD1 therapy when compared to control Ig treated mice (FIG. 19G). To understand the clinical relevance, presence of mast cells was confirmed in tumor sections and in analysis of two independent data sets of melanoma patients receiving anti-PD1 therapy (FIGS. 19H and 19I; FIG. 26). For the mast cells to be recruited by tumor cells, they need chemokines as chemo-attractants and higher transcription of several chemokine genes (CCL2, CCL3, CCL4, CCLS, CXCL9, CXCL10 and CXCL11) was observed after anti-PD1 treatment (FIG. 27A). Of note, is the presence of CXCL10 that melanomas are known to secrete, and its presence was confirmed (FIG. 27B). Mast cells express several chemokine receptors including CXCR2 and CXCR3 (FIGS. 27C, 27D). CXCL10 are known to bind CXCR3 that are present on mast cells resulting in their infiltration in high numbers in the tumor area. Further examination of the tumor tissue sections after anti-PD1 therapy revealed the co-localization of mast cells and FOXP3+ Treg cells (FIG. 19J). Without wishing to be bound by theory, this suggested a cross talk between these two cell types that may have resulted in downmodulation of HLA class I on tumor cells (FIG. 19D and FIG. 27E). If mast cells contribute to therapy resistance of anti-PD1 treatment, then depletion of these cells should result in tumor regression. Mast cells are known to be c-kit receptor positive and one can target these cells by pharmacological intervention using drugs that can inhibit the c-kit receptor. Sunitinib, a multi-targeted receptor tyrosine kinase inhibitor with targets including c-kit receptor, was used, and it was followed with anti-PD1 therapy in treating established tumors in Hu-mice. Inclusion of Sunitinib in combination with anti-PD1 caused complete regression of tumors in 3/5 mice while, treatment with Sunitinib alone did not influence the tumor growth significantly (FIGS. 19K and 19L). Hu-mice that showed complete regression of tumors showed no signs of recurrence for 4 weeks after cessation of therapy and all the Hu-mice were able to reject re-challenged tumors suggestive of memory T-cell responses. Our results suggest identification of a new resistance mechanism that is dependent on tumor infiltrating mast cells.

Tumors play a dynamic role in evading therapy responses. This is done either directly or indirectly by enlisting the help of tumor stromal cells in therapy resistance. Several known mechanisms have been identified and some of them include, alteration and/or activation of redundant signaling pathways, and downmodulation of the antigen presenting machinery to evade anti-tumor specific T-cells, all contributing to resurgence of resistant tumor cells. We and others have shown that tumor-infiltrating fibroblasts, macrophages and B-cells play an important role in therapy resistance.

Mast cells were shown to play a unique role in downmodulating the immune response to anti-PD1 therapy (FIG. 19M). There is an increase in chemokine production causing increased infiltration of mast cells into the tumor after anti-PD1 therapy. Co-localization of mast cells and FOXP3+ T-reg cells was observed in selective areas of the tumor sections suggesting localized pockets of resistance. The cross-talk between FOXP3+ T-reg cells and mast cells then resulted in downmodulation of HLA-class I molecules in tumors. Lack of HLA-class I on melanoma cells resulted in poor infiltration of CD45RO+, CD8+, Granzyme B+ T-cells and negligible tumor cell lysis causing therapy resistance. The combination of Sunitinib and anti-PD1 resulted in complete regression of tumors. Without wishing to be bound by theory, this result suggests that depletion of mast cells is beneficial to immune checkpoint therapy responses.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A humanized mouse comprising:
(a) CD34+ cells from human fetal liver and/or human fetal thymus, and
(b) one or more exogenously introduced polynucleotides encoding a cytokine or cytokine receptor.

2. The mouse of claim 1, wherein the mouse is an immunodeficient mouse.

3. The mouse of claim 2, wherein the mouse is an NSG™ mouse.

4. The mouse of claim 1, wherein the mouse comprises subpopulations of human hematopoietic cells induced following expression of a cytokine or cytokine receptor from the one or more exogenously introduced polynucleotides.

5. The mouse of claim 4, wherein the subpopulations of human hematopoietic cells comprise T cells, B cells, NK cells, monocytes, dendritic cells, or combinations thereof

6. The mouse of claim 1, wherein the one or more exogenously introduced polynucleotides encodes a cytokine.

7. The mouse of claim 1, wherein the cytokine or cytokine receptor is human, mouse, or combinations thereof.

8. The mouse of claim 7, wherein the cytokine or cytokine receptor is recombinant.

9. The mouse of claim 6, wherein the cytokine is Colony stimulating factor 2 (CSF2), Interleukin-3 (IL3), Interleukin-7 (IL7), Stem cell factor (SCF), Fms Related Tyrosine Kinase 3 (FLT3), Thrombopoietin (TPO), Colony stimulating factor 1 (CSF1), Colony stimulating factor 3 (CSF3), Erythropoietin (EPO), Interleukin-15 (IL15), c-kit, or combinations thereof.

10. A method of generating a humanized mouse, the method comprising:
transplanting CD34+ cells from human fetal liver and/or human fetal thymus into an immunodeficient mouse; and
delivering one or more polynucleotides encoding a cytokine or cytokine receptor to the mouse, thereby generating the humanized mouse.

11. The method of claim 10, wherein the one or more polynucleotides encodes a cytokine.

12. The method of claim 10, wherein when more than one polynucleotides are delivered, the more than one polynucleotides are delivered to the mouse simultaneously or serially.

13. The method of claim 10, wherein when the cytokine or cytokine receptor is expressed in the humanized mouse from the one or more polynucleotides, subpopulations of human hematopoietic cells are generated.

14. The method of claim 10, wherein the mouse is a NSGTM mouse.

15. The method of claim 13, wherein the subpopulations of human hematopoietic cells comprise T cells, B cells, NK cells, monocytes, dendritic cells, or combinations thereof.

16. The method of claim 10, wherein the cytokine or cytokine receptor is human, mouse, or combinations thereof.

17. The method of claim 16, wherein the cytokine or cytokine receptor is recombinant.

18. The method of claim 11, wherein the cytokine is Colony stimulating factor 2 (CSF2), Interleukin-3 (IL3), Interleukin-7 (IL7), Stem cell factor (SCF), Fms Related Tyrosine Kinase 3 (FLT3), Thrombopoietin (TPO), Colony stimulating factor 1 (CSF1), Colony stimulating factor 3 (CSF3), Erythropoietin (EPO), Interleukin-15 (IL15), c-kit, or combinations thereof.

19. The method of claim 10, wherein the one or more polynucleotides encoding a cytokine or cytokine receptor are delivered in a plasmid or vector.

20. The method of claim 19, wherein the vector is a viral vector.

21. The mouse of claim 1, wherein the cytokine or cytokine receptor is under the control of a constitutive promoter.

22. The mouse of claim 21, wherein the constitutive promoter is a CMV promoter.

23. A method for generating a humanized mouse melanoma model comprising:
generating a humanized mouse by the method of claim 10; and
transplanting HLA-A allele matched melanoma cells into the humanized mouse.

24. A method for measuring an immune response to a melanoma cell comprising:
administering HLA-A allele matched melanoma cells into the humanized mouse of claim 1; and
measuring an immune response to the melanoma cells in the humanized mouse.

25. A method for testing a vaccine comprising:
administering a vaccine to the humanized mouse of claim 1; and
measuring an immune response to the vaccine in the humanized mouse.

26. The method of claim 25, wherein the vaccine is human telomerase reverse transcriptase (TERT).

27. A method for testing a drug or treatment in a humanized mouse comprising:
administering the drug or treatment to the humanized mouse of claim 1; and
measuring an immune response to the drug or treatment in the humanized mouse.

28. The method of claim 27, further comprising measuring the effectiveness of the drug or treatment in the humanized mouse.

29. The method of claim 27, wherein the treatment comprises immune checkpoint inhibitory therapy.

30. The method of claim 29, wherein the immune checkpoint inhibitory therapy comprises administration of anti-PD1 antibody or anti-PDL1 antibody to the humanized mouse.

31. A method for synthesizing a polypeptide in a humanized mouse comprising:
administering an exogenous polynucleotide encoding the polypeptide to the humanized mouse of claim 1.

32. The method of claim 31, further comprising collecting or detecting the polypeptide.

33. The method of claim 31, wherein the polypeptide is an antibody or antibody fragment.

34. The method of claim 33, wherein the antibody is a monoclonal antibody or fragment thereof.

35. The method of claim 33, wherein the antibody or antibody fragment is a Fv, Fab, F(ab)₂, or a single chain antibody (scFv).

36. The method of claim 33, wherein the antibody or antibody fragment is a chimeric, human or humanized antibody or antibody fragment.
